(19) United States
(12) Patent Application Publication
(10) Pub. No.: US 2026/0007405 A1
(43) Pub. Date: Jan. 8, 2026

(54) END TOOL AND SURGICAL INSTRUMENT

(71) Applicant: LIVSMED INC., Seongnam-si (KR)

(72) Inventor: Woo Jung CHOI, Siheung-si (KR)

(21) Appl. No.: 19/260,050

(22) Filed: Jul. 3, 2025

(30) Foreign Application Priority Data
Jul. 5, 2024 (KR) 10-2024-0088926

(51) Int. Cl. A61B 17/072

(52) U.S. Cl. CPC A61B 17/07207; A61B 2017/07271

(57) ABSTRACT

Provided are an end tool and a surgical instrument. The end tool includes a first jaw configured to be rotatable around a pitch rotation shaft and including a cartridge accommodation part configured to accommodate a cartridge therein, a second jaw facing the first jaw and configured to perform a relative rotational motion with respect to the first jaw, and the cartridge accommodated in the cartridge accommodation part and including a plurality of staples, wherein the second jaw includes guard parts extending toward the first jaw, such that at least one region of each of the guard parts is positioned in the cartridge accommodation part during the relative rotational motion of the second jaw with respect to the first jaw.

### CROSS-REFERENCE TO RELATED APPLICATION

This application is based on and claims priority under 35 USC § 119 to Korean Patent Application No. 10-2024-0088926, filed on Jul. 5, 2024, in the Korean Intellectual Property Office, the disclosure of which is incorporated by reference herein in its entirety.

### BACKGROUND

### 1. Field

The present disclosure relates to an end tool and a surgical instrument.

### 2. Description of the Related Art

In recent years, laparoscopic surgery has been actively used to reduce postoperative recovery time and complications through small incisions. The laparoscopic surgery is a surgical method in which a plurality of small holes are drilled in the abdomen of a patient and the inside of the abdominal cavity is observed through these holes, and is widely used in general surgery and the like.

In performing such laparoscopic surgery, various instruments are being used. For example, suturing instruments that are inserted into the body to suture the surgical site within the abdominal cavity are used, and a surgical stapler that uses medical staples to suture the surgical site is used as the suturing instrument.

In general, a surgical stapler is a medical instrument that is often used for cutting and anastomosis of an organ in abdominal and thoracic surgery. The surgical stapler includes an open stapler used in thoracotomy and laparotomy and an endo stapler used in thoracoscopic surgery and celioscopic surgery.

The surgical stapler has advantages of not only shortening operation time since cutting of a surgical site and anastomosis of an organ are simultaneously performed, but also accurately stapling the surgical site. In addition, the surgical stapler has advantages of a faster recovery and a smaller scar than those when tissue is cut and stapled using a surgical stapling thread, and thus has been widely used in modern surgical operations. In particular, the surgical stapler has been widely used in cancer surgery to cut cancer tissue and suture a cut site.

The background art described above is technical information retained by the present inventors in order to derive the present disclosure or obtained by the present inventors in the process of deriving the present disclosure, and thus is not necessarily known art disclosed to the general public before the filing of the present application.

### SUMMARY

The present disclosure is directed to providing an end tool and a surgical instrument enabling safe stapling procedures, the surgical instrument being mountable on a robotic arm or manually operable for use in laparoscopic surgery or various other surgical procedures.

According to an aspect of the present disclosure, there is provided an end tool including a first jaw configured to be rotatable around a pitch rotation axis and including a cartridge accommodation part configured to accommodate a cartridge therein, a second jaw facing the first jaw and configured to rotate with respect to the first jaw, and the cartridge accommodated in the cartridge accommodation part and including a plurality of staples, wherein the second jaw includes a guard part extending toward the first jaw, such that at least one region of the guard part is positioned in the cartridge accommodation part during the rotation of the second jaw with respect to the first jaw.

In an embodiment of the present disclosure, the first jaw may further include side wall parts extending from opposite sides of a base of the first jaw toward the second jaw, and the side wall parts may form the cartridge accommodation part in a space therebetween.

In an embodiment of the present disclosure, the guard part may be positioned between the cartridge and the side wall parts when the cartridge is accommodated in the cartridge accommodation part.

In an embodiment of the present disclosure, the guard part comprises a first guard part and a second guard part, the first guard part and the second guard part are disposed to face each other on opposite sides of the second jaw with respect to a longitudinal axis of the second jaw, and a width between the first guard part and the second guard part is less than a width between the side wall parts.

In an embodiment of the present disclosure, a width between the side wall parts may be constant in a direction in which the first jaw extends.

In an embodiment of the present disclosure, the guard part may be positioned closer to the cartridge than to the side wall parts when the cartridge is accommodated in the cartridge accommodation part.

In an embodiment of the present disclosure, the guard part may be positioned on a proximal side of the second jaw.

In an embodiment of the present disclosure, at least one region of the guard part is positioned in the cartridge accommodation part when the first jaw and the second jaw are spaced apart at the greatest distance.

An embodiment of the present disclosure, the end tool may further include a wire positioned in the cartridge accommodation part in a longitudinal direction of the first jaw, wherein the guard part may be positioned between the cartridge and the wire when the cartridge is accommodated in the cartridge accommodation part.

In an embodiment of the present disclosure, the second jaw may further include a hinge part having a first rotation shaft serving as a center of the rotation with respect to the first jaw, and the guard part may be configured to cover at least a portion of outer side of the hinge part.

In an embodiment of the present disclosure, the guard part may restricts a body tissue from being inserted into a predefined region when the body tissue is clamped between the first jaw and the second jaw.

In an embodiment of the present disclosure, in a closed state of the second jaw, in which the second jaw is rotated to be in closest proximity to the first jaw, a width of the first jaw may define a maximum outer diameter of the end tool, and a width of the second jaw may be less than the maximum outer diameter of the end tool.

In an embodiment of the present disclosure, the guard part comprises a first guard part and a second guard part, and the width of the first jaw may be greater than a distance between the first guard part and the second guard part in at least one region.

In an embodiment of the present disclosure, in the closed state of the second jaw, the guard part may be restricted from coming into contact with an outside by the first jaw.

In an embodiment of the present disclosure, in a closed state in which the first jaw and the second jaw are rotated to be in closest proximity to each other, a maximum outer diameter of the end tool may be defined as a maximum distance between the first jaw and the second jaw in a cross section perpendicular to the longitudinal axis of the end tool, and the guard parts may be positioned in a space defined by the maximum outer diameter of the end tool.

According to an aspect of the present disclosure, there is provided a surgical instrument including a connection part, an end tool disposed on a first side of the connection part and rotatable in at least one direction, and a manipulation part disposed on a second side of the connection part and configured to control a rotation of the end tool, wherein the end tool includes a first jaw configured to be rotatable around a pitch rotation axis and including a cartridge accommodation part configured to accommodate a cartridge therein, a second jaw facing the first jaw and configured to rotate with respect to the first jaw, and the cartridge accommodated in the cartridge accommodation part and including a plurality of staples, wherein the second jaw includes a guard part extending toward the first jaw, such that at least one region of the guard part is positioned in the cartridge accommodation part during the rotation of the second jaw with respect to the first jaw.

In an embodiment of the present disclosure, the first jaw may further include side wall parts extending from opposite sides of a base of the first jaw toward the second jaw, and the side wall parts may form the cartridge accommodation part in a space therebetween.

In an embodiment of the present disclosure, the guard part may be positioned between the cartridge and each of the side wall parts when the cartridge is accommodated in the cartridge accommodation part.

In an embodiment of the present disclosure, in a closed state of the second jaw, in which the second jaw is rotated to be in closest proximity to the first jaw, a width of the first jaw may define a maximum outer diameter of the end tool, and a width of the second jaw may be less than the maximum outer diameter of the end tool.

In an embodiment of the present disclosure, in a closed state in which the first jaw and the second jaw are rotated to be in closest proximity to each other, a maximum outer diameter of the end tool may be defined as a maximum distance between the first jaw and the second jaw in a cross section perpendicular to the longitudinal axis of the end tool, and the guard part may be positioned in a space defined by the maximum outer diameter of the end tool.

Other aspects, features, and advantages other than those described above will become apparent from the following drawings, claims, and detailed description of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIGS. 1A to 1C are views for comparing and describing a structure of an end tool according to an embodiment of the present disclosure with that of a conventional end tool;

FIG. 2 is a perspective view illustrating the end tool in an open state according to an embodiment of the present disclosure;

FIG. 3 is a perspective view illustrating the end tool in a closed state according to an embodiment of the present disclosure;

FIG. 4 is a perspective view illustrating the end tool of FIG. 2 with a housing of a first jaw removed;

FIG. 5 is an exploded perspective view illustrating the first jaw and a second jaw of the end tool of FIG. 2.

FIG. 6 is a perspective view illustrating the second jaw of the end tool of FIG. 2;

FIG. 7 is a cross-sectional view illustrating the end tool of FIG. 2, in which the first jaw and the second jaw are disassembled;

FIG. 8 is a cross-sectional view taken along line A-A′ of FIG. 2;

FIG. 9 is a cross-sectional view taken along line B-B′ of FIG. 3;

FIG. 10 is a view for describing guard parts and hinge parts of the end tool of FIG. 3;

FIG. 11 is a bottom view illustrating the second jaw of the end tool of FIG. 10;

FIG. 12 is a view for describing functions of the guard parts of the end tool according to an embodiment of the present disclosure;

FIG. 13 is a perspective view illustrating a reload assembly to which the end tool according to an embodiment of the present disclosure is applied;

FIG. 14 is a perspective view illustrating a surgical instrument to which the end tool according to an embodiment of the present disclosure is applied;

FIG. 15 is a schematic perspective view of a surgical instrument according to another embodiment of the present disclosure;

FIG. 16 is a schematic perspective view for describing an end tool illustrated in FIG. 15;

FIG. 17 is a perspective view of the end tool of FIG. 16 viewed from another direction;

FIG. 18 is a schematic perspective view of the end tool of FIG. 16 with a second jaw removed;

FIG. 19 is a schematic perspective view of the end tool of FIG. 18 with a cartridge removed;

FIG. 20 is a transparent perspective view of FIG. 19;

FIG. 21 is a perspective view schematically illustrating the second jaw of the end tool of FIG. 16.

FIG. 22 is a perspective view schematically illustrating a first jaw of the end tool of FIG. 16;

FIG. 23 is a plan view schematically illustrating the first jaw of the end tool of FIG. 16;

FIG. 24 is a perspective view illustrating an operation member of the end tool of FIG. 16.

FIG. 25 is a perspective view of the operation member of FIG. 24 viewed from another direction;

FIG. 26 is a front view of the operation member of FIG. 24 viewed in one direction;

FIG. 27 is a schematic perspective view illustrating a portion of the end tool of FIG. 16;

FIG. 28 is a front view of FIG. 27 viewed in one direction;

FIG. 29 is a schematic plan view for describing the operation member, a fixed pulley, and a forward-moving wire of the end tool of FIG. 20.

FIG. 30 is a schematic perspective view for describing the operation member, the fixed pulley, and the forward-moving wire of the end tool of FIG. 16;

FIG. 31 and FIGS. 32A to 32C are schematic views for describing an operation of the operation member of the end tool of FIG. 16;

FIG. 33 is a perspective view illustrating the first jaw and the cartridge of the surgical instrument of FIG. 15; and

FIGS. 34 and 35 are views for describing a switching pulley, a yaw pulley, and a pitch pulley of the end tool of the surgical instrument of FIG. 15.

### DETAILED DESCRIPTION

Hereinafter, the following embodiments will be described in detail with reference to the accompanying drawings. When describing with reference to the drawings, identical or corresponding components will be assigned the same reference numerals and duplicate descriptions thereof will be omitted.

Since various transformations can be made to these embodiments, specific embodiments will be illustrated in the drawings and described in detail in the detailed description. The effects and features of the present embodiments and the accompanying methods thereof will become apparent from the following description of the contents, taken in conjunction with the accompanying drawings. However, the present embodiments are not limited to the embodiments disclosed below, but may be implemented in various forms.

In describing the present disclosure, a detailed description of known related arts will be omitted when it is determined that the gist of the present disclosure may be unnecessarily obscured.

In the following embodiments, singular forms are intended to include plural forms as well, unless the context clearly indicates otherwise. Although terms such as “first,” “second,” and the like may be used to describe various components, such components should not be limited to the above terms The terms are only used to distinguish one component from another.

In the following embodiments, terms such as “include” or “have” means that the features or components described in the specification are present, and the possibility that one or more other features or components will be added is not excluded in advance.

In the following embodiments, when a unit, region, or component is referred to as being formed on another unit, region, or component, it can be directly formed on the other unit, region, or component. That is, for example, intervening units, regions, or components may be present.

In the following embodiments, terms such as “connecting” or “coupling” two members do not necessarily mean a direct and/or fixed connection or coupling of the two members, unless the context clearly indicates otherwise, and do not preclude another members from being interposed between the two members.

Sizes of components in the drawings may be exaggerated or reduced for convenience of description. For example, since the size and thickness of each component shown in the drawings are arbitrarily illustrated for convenience of description, the following embodiments are not necessarily limited thereto.

An end tool according to an embodiment of the present disclosure is directed to a surgical instrument that is mountable on a robot arm or manually operable for use in laparoscopic surgery or various other surgical procedures. Specifically, the end tool according to an embodiment of the present disclosure is directed to a laparoscopic stapler and is configured to facilitate safe stapling procedures.

A laparoscopic stapler performs stapling by inserting body tissue between an anvil and a lower jaw, followed by clamping and firing operations.

The stapler includes a cartridge in which staple pins are positioned and a knife, wherein the knife moves in a longitudinal direction of the jaw to cut body tissue, and simultaneously, the staple pins are ejected toward the body tissue to clamp the body tissue.

In this case, a region in which cutting and suturing are performed may be determined by a firing start point and a firing end point. That is, an actual region in which the procedure is performed may be determined by the firing start point and the firing end point. Specifically, the region in which suturing is performed is limited to a region in which the staple pins are positioned in the cartridge, and thus, may be different from a region in which the cutting of body tissue is performed.

Accordingly, during insertion of the body tissue between the anvil and the jaw, when the tissue is pushed deeply beyond the starting point of the region, in which the suturing is performed, a region may arise in which the tissue is cut but not sutured.

To prevent this, stapler products have a structure that blocks body tissue from being inserted beyond the region, in which suturing is performed, in a space between the anvil and the jaw.

According to the related art, a tissue guard corresponding to such a structure is attached to the anvil, and when the anvil and the lower jaw are closed, the tissue guard covers an outer side surface of the lower jaw.

Meanwhile, a device known as a trocar is used to insert laparoscopic surgical tools into the patient's body, and as a diameter of the surgical tool increases, a size of the trocar also increases, which may lead to a larger incision at the surgical site.

The trocar used with laparoscopic staplers is typically a 12 mm-grade trocar with a diameter ranging from 12 mm to 12.9 mm, and in order to enable the stapler to be used with such standard trocars, the size of the end tool may be restricted.

For example, when the tissue guard of the anvil is structured to cover the outer side surface of the lower jaw, the width of the anvil increases as the width of the lower jaw increases, which may ultimately lead to an enlargement of the diameter of the end tool. For example, an end tool having the structure described above may have a diameter exceeding 13 mm, which could make it difficult to use with existing trocars. That is, an increase in the size of the surgical tool may result in restrictions on the use of standard trocars.

An end tool according to an embodiment of the present disclosure may resolve the above-described issues through a structure different from that of the end tool of the related art. This will be described in detail with reference to the drawings.

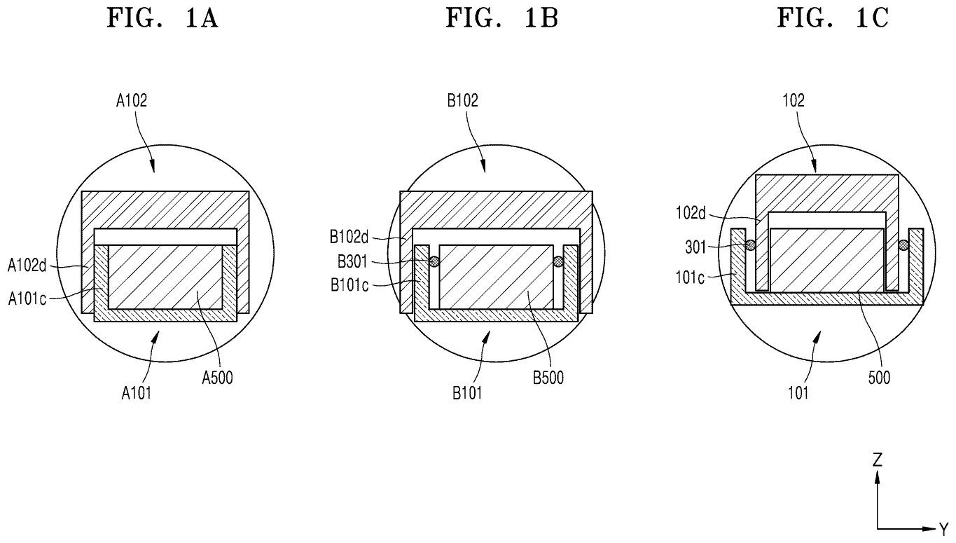

FIGS. 1A to 1C are views for comparing and describing a structure of an end tool according to an embodiment of the present disclosure with that of a conventional end tool. Specifically, FIG. 1A is a schematic view of an end tool according to a comparative example viewed from the front, and FIG. 1B is a schematic view of an end tool according to another comparative example, which has a lower jaw designed with an increased width and a tissue guard covering an outer side portion of the jaw. In addition, FIG. 1C is a schematic view of an end tool according to an embodiment of the present disclosure viewed from the front.

Referring to FIG. 1A, a lower jaw A101 is formed in the shape of a housing including side wall parts A101c and may accommodate a cartridge. In addition, a tissue guard may be coupled to an anvil A102, and for example, guard parts A102d may be coupled to the anvil A102. The guard parts A102d are disposed in a shape that covers outer side portions of the side wall parts A101c of the lower jaw A101.

As shown in FIG. 1B, the end tool may be designed to have cases in which wires B301 are positioned on opposite sides of a cartridge B500. That is, by adopting a structure in which the wires B301 are positioned between the cartridge B500 and side wall parts B101c of a lower jaw B101, a width of the lower jaw B101 may be increased compared with a conventional design. Alternatively, the width of the lower jaw B101 may be increased due to other design factors. In this case, when a tissue guard attached to an anvil B102 has a structure that covers an outer side surface of the lower jaw B101, a width between guard parts B102d may increase, and the overall width of the anvil B102 may also increase. An increase in the overall diameter of the end tool as described above may restrict the use of trocars with the conventional end tools and lead to larger incisions at the surgical site due to the requirement for larger trocars.

On the other hand, as shown in FIG. 1C, in the end tool according to an embodiment of the present disclosure, guard parts 102d of a tissue guard may be positioned in a housing of a lower jaw 101. Accordingly, the end tool according to an embodiment of the present disclosure can be inserted into a standard trocar, such as a 12 mm-class trocar, while securing paths for wires 301 positioned on the lower jaw 101.

Hereinafter, the end tool according to an embodiment of the present disclosure will be described in detail with reference to the drawings.

In describing the present disclosure, the part closer to a user side, i.e., the part closer to a manipulation part, will be described as a proximal end, and the part farther from the user side, that is, the part closer to an end portion of an end tool 100, will be described as a distal end.

For example, the part of the end tool 100 closer to a connection part (or shaft) 400 will be defined as a proximal end 100p, and the part farther from the connection part (or shaft) 400, that is, the part closer to the end portion of the end tool 100 will be defined and described as a distal end 100d.

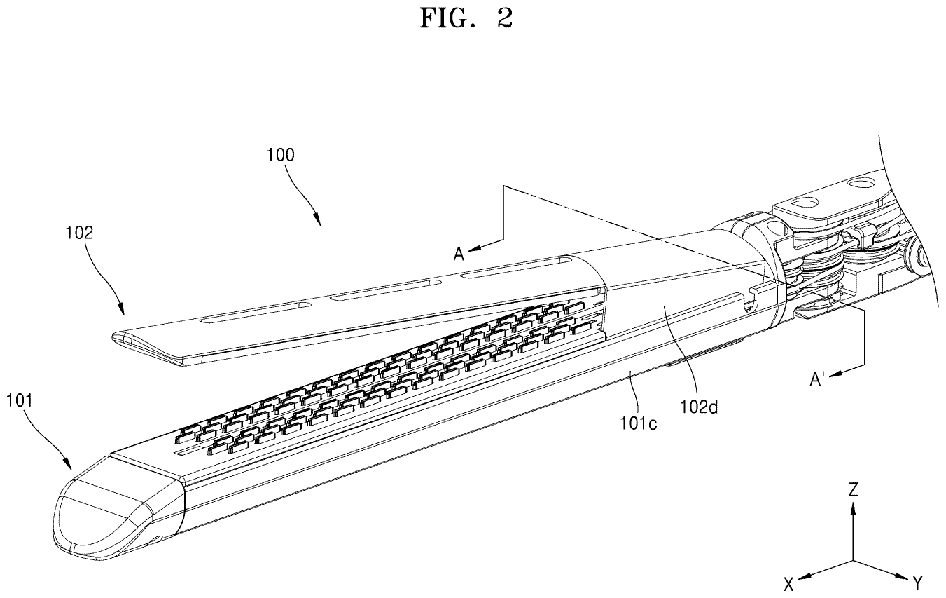

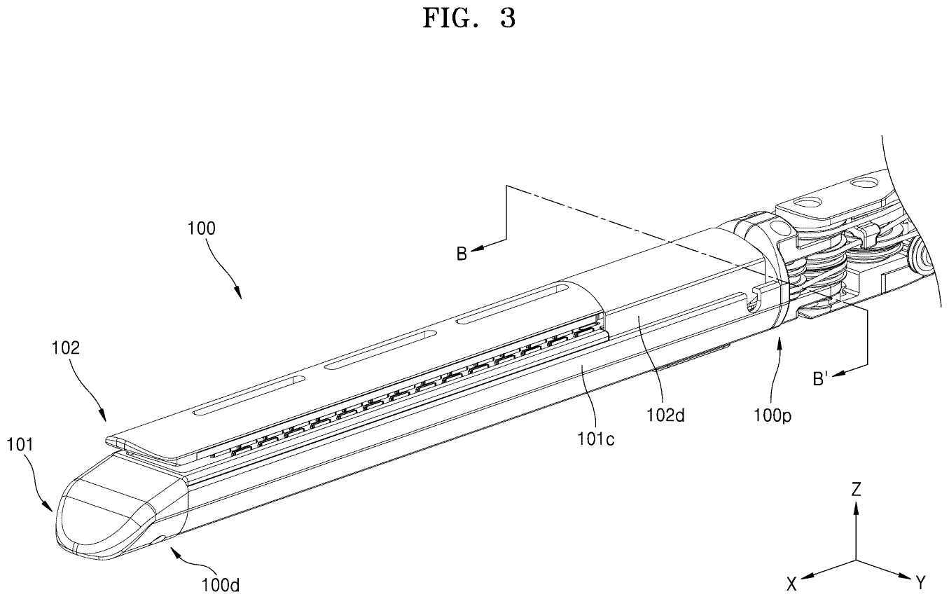

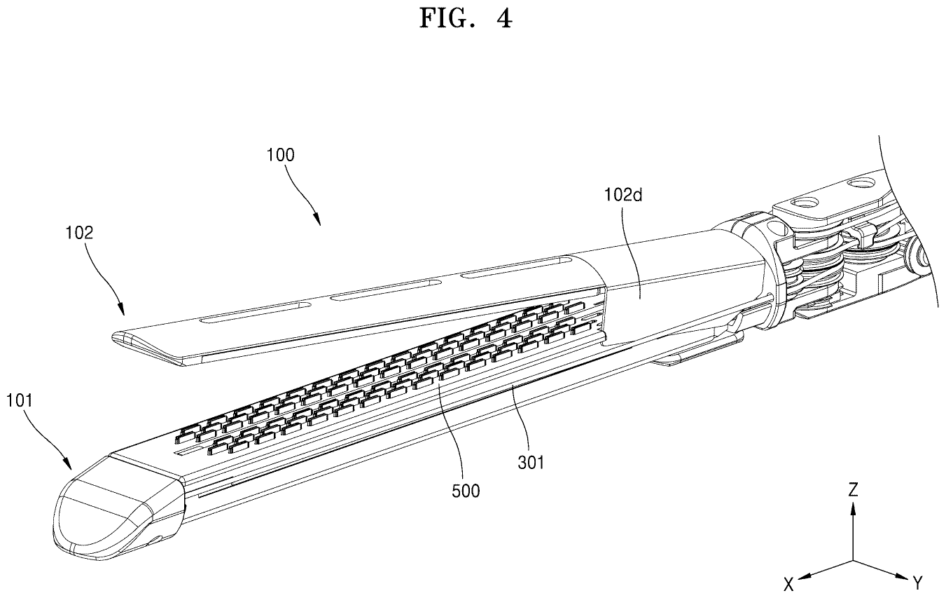

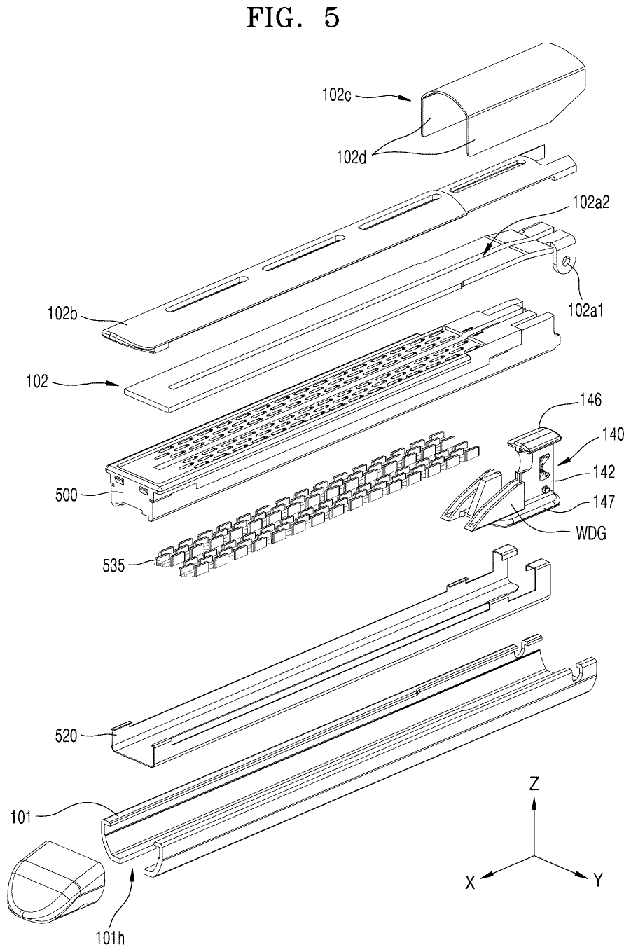

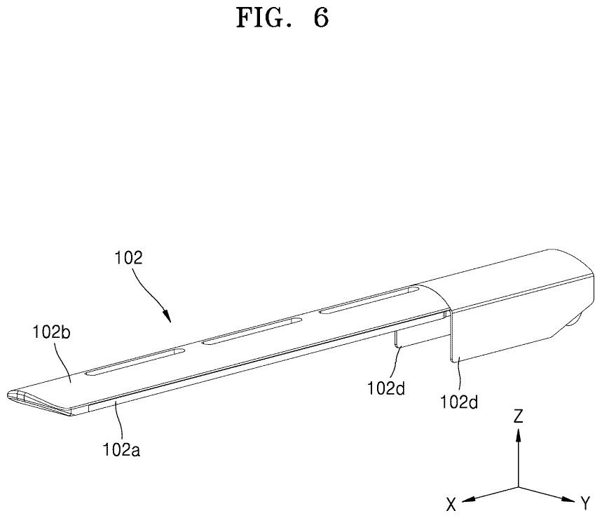

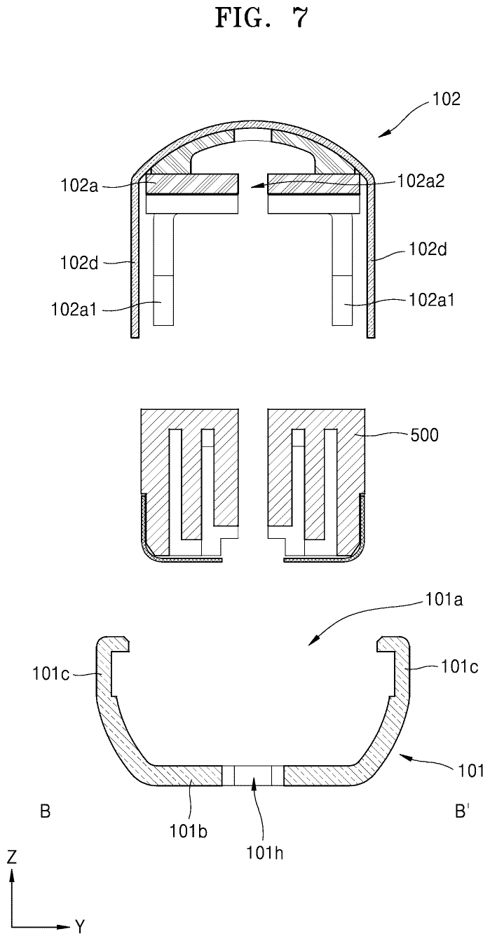

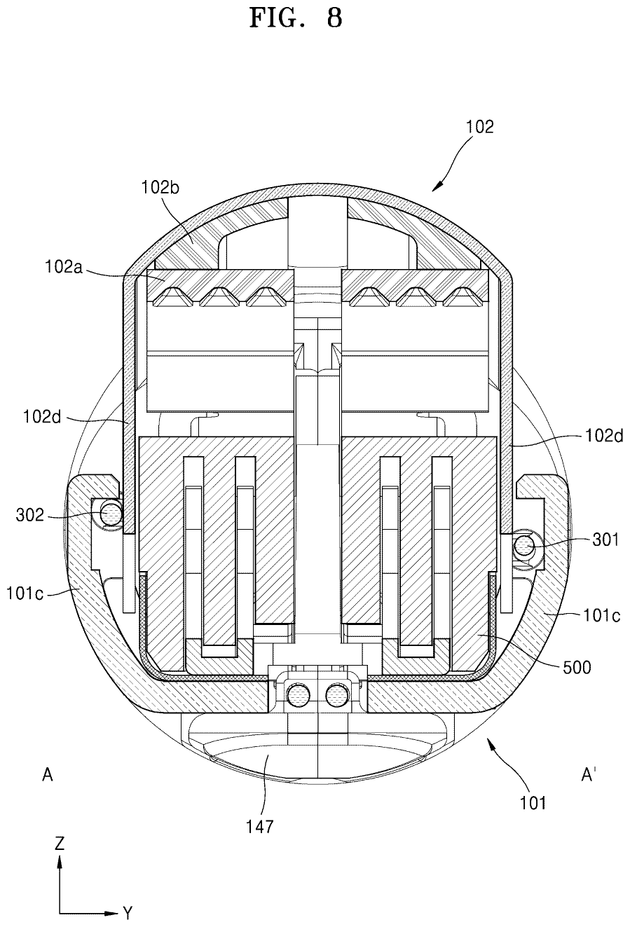

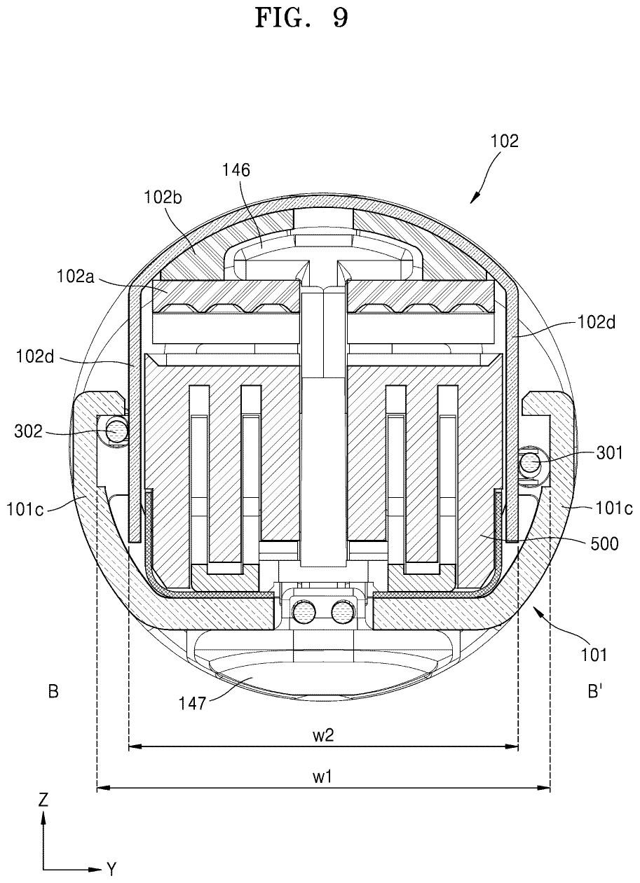

FIG. 2 is a perspective view illustrating the end tool in an open state according to an embodiment of the present disclosure, and FIG. 3 is a perspective view illustrating the end tool in a closed state according to an embodiment of the present disclosure. FIG. 4 is a perspective view illustrating the end tool of FIG. 2 with a housing of a first jaw removed. FIG. 5 is an exploded perspective view illustrating the first jaw and a second jaw of the end tool of FIG. 2, and FIG. 6 is a perspective view illustrating the second jaw of the end tool of FIG. 2. FIG. 7 is a cross-sectional view illustrating the end tool of FIG. 2, in which the first jaw and the second jaw are disassembled. FIG. 8 is a cross-sectional view taken along line A-A′ of FIG. 2, and FIG. 9 is a cross-sectional view taken along line B-B′ of FIG. 3.

The end tool according to an embodiment of the present disclosure may be connected to one end portion of the connection part (shaft) of a surgical instrument and inserted into a surgical site to perform motions required for surgery.

Referring to FIGS. 2 to 9, the end tool according to an embodiment of the present disclosure may include a first jaw, a second jaw, and a cartridge.

A first jaw 101 may be configured to be rotatable around a pitch rotation shaft, and a second jaw 102 facing the first jaw 101 may be configured to be rotatable relative to the first jaw 101.

From another perspective, the first jaw 101 and the second jaw 102 may be formed to face each other and configured to perform opening and closing motions. In other words, the first jaw 101 and the second jaw 102 may move toward and away from each other. In addition, the first jaw 101 and the second jaw 102 may be configured to rotate together around one shaft. For example, the first jaw 101 and the second jaw 102 can perform a pitch or yaw rotational motion around an extension direction of the shaft.

A cartridge 500 may be disposed to be accommodated in the first jaw 101, and a plurality of staples are positioned inside the cartridge 500. When an operation member 140 receives a force through a forward-moving wire while the first jaw 101 and the second jaw 102 are close to each other, such as when the first jaw 101 and the second jaw 102 are closed with body tissue interposed therebetween, the operation member 140 may push and raise staples while moving toward the distal end 100d of the first jaw 101, thereby performing stapling. At this point, one or more clamps of the operation member 140 may protrude to the outside of the first jaw 101 or the second jaw 102, allowing the operation member 140 to move forward while applying pressure to an outer surface of the first jaw 101 or the second jaw 102, which facilitates the smooth progression of a stapling process.

Meanwhile, the operation member 140 may be used together with a wedge WDG. For example, the wedge WDG may be prepared separately from the operation member 140 and then disposed adjacent to the operation member 140 in the first jaw 101. In addition, as another example, the operation member 140 and the wedge WDG may be integrally formed as one body. The wedge WDG may be positioned on at least one side of a body and may be formed to have a predetermined inclined surface. That is, the wedge WDG may be formed to be inclined by a certain degree with respect to an extension direction of the end tool 100. In other words, the wedge WDG may be formed to have a greater height at the proximal end 100p side of the first jaw 101 than the distal end 100d side of the first jaw 101.

The wedge WDG may be formed to be sequentially in contact with withdrawal members 535 or a plurality of staples positioned inside the cartridge 500 and may serve to sequentially push and raise the staples. Further details will be described below.

The first jaw 101 may be formed in an elongated bar shape as a whole. The cartridge 500 may be accommodated in the first jaw 101 at the distal end 100d side, and a rotation shaft may be positioned in the proximal end 100p so that the first jaw 101 is rotationally movable therearound.

The first jaw 101 may include a cartridge accommodation part 101a that accommodates the cartridge 500. Specifically, the first jaw 101 may be formed entirely in the form of a hollow box with one surface (upper surface) thereof is removed, such that the cartridge accommodation part 101a capable of accommodating the cartridge 500 may be formed inside the first jaw 101. That is, the first jaw 101 may be formed in an approximately “U” shape in cross section.

Specifically, the first jaw 101 may include a base 101b and side wall parts 101c formed to extend from the base 101b toward the second jaw 102. That is, the side wall parts 101c may be formed to face each other on opposite sides of the base 101b, which is formed to extend in a longitudinal direction of the first jaw 101, and the cartridge accommodation part 101a may be formed in a space between the side wall parts 101c.

Meanwhile, a width between the side wall parts 101c may be constant in a direction in which the first jaw extends 101.

In other words, a width of the cartridge accommodation part 101a formed in the longitudinal direction of the first jaw 101 may be constant in the longitudinal direction of the first jaw 101.

A guide groove 101h may be formed in the base 101b of the first jaw 101, i.e., a bottom surface opposite to an upper open region formed by removing one surface. Specifically, the guide groove 101h may be configured to guide a linear motion of the operation member 140.

The guide groove 101h may be configured to guide the operation member 140, and may have a through-hole shape in a region facing the operation member 140. Through this, one region of the operation member 140, such as at least one region of a body 142 of the operation member 140, or a second clamp 147 connected thereto may pass through the guide groove 101h to exit to the outside of the first jaw 101. When the operation member 140 moves forward, the second clamp 147 may pass through the guide groove 101h of the first jaw 101 to be exposed to the outside of the first jaw 101, and may come into contact with a lower surface of the first jaw 101 or apply pressure thereto. As the operation member 140 moves, the second clamp 147 applies pressure on the lower surface of the first jaw 101 and a first clamp 146 applies pressure on an upper surface of the second jaw 102 such that a gap between the second jaw 102 and the first jaw 101 decreases, allowing the second jaw 102 to naturally remain in a closed state with respect to the first jaw 101.

The second jaw 102 may be formed in an elongated bar shape as a whole, and for example, the second jaw 102 may be formed in a rod shape to correspond to the first jaw 101 in at least one region. An anvil is formed in the second jaw 102 at the distal end 100d side, and a region coupled to the first jaw 101 may be included in the proximal end 100p of the second jaw 102. As an example, the second jaw 102 may be configured to be rotatable around one shaft of the proximal end 100p with respect to the first jaw 101.

As a specific example, among surfaces of the second jaw 102, the surface facing the first jaw 101 may have an anvil 102a formed thereon, may be formed in a flat plane shape, and may have shapes corresponding to the shapes of the staples to be described below. The anvil 102a of the second jaw 102 may serve as a support for holding the staple on the opposite side of the operation member 140 when the operation member 140 pushes and raises the staple during a stapling motion, thereby bending the staple.

The second jaw 102 includes a guide groove 102a2. The guide groove 102a2 may have a shape elongated in a longitudinal direction of the second jaw 102.

The guide groove 102a2 may be configured to guide the operation member 140 and may have a through-hole shape in a region facing the operation member 140. Through this, one region of the operation member 140, such as at least one region of the body 142 of the operation member 140, or the first clamp 146 connected thereto may pass through the guide groove 102a2 to exit to the outside of the second jaw 102. When the operation member 140 moves forward, the first clamp 146 may pass through the guide groove 102a2 of the second jaw 102 to be exposed to the outside of the second jaw 102, and may come into contact with the upper surface of the second jaw 102 or apply pressure thereto. As the operation member 140 moves, the first clamp 146 applies pressure on the upper surface of the second jaw 102 and the second clamp 147 to be described below applies pressure on the lower surface of the first jaw 101 such that the gap between the second jaw 102 and the first jaw 101 decreases, allowing the second jaw 102 to naturally remain in a closed state with respect to the first jaw 101.

The second jaw 102 of the end tool 100 according to an embodiment of the present disclosure may include guard parts 102d.

The guard parts 102d may be plate-shaped members formed to extend from the second jaw 102. Specifically, the guard parts 102d may be formed in a shape that laterally covers a separation space existing between the first jaw 101 and the second jaw 102 when the first jaw 101 and the second jaw 102 are open.

Specifically, the guard parts 102d may be formed to face each other on opposite sides of a longitudinal axis of the second jaw 102. In addition, the guard parts 102d may be positioned on the second jaw 102 at the proximal end 100p side.

In other words, the guard parts 102d may be positioned to be adjacent to a rotation shaft of the second jaw 102.

In an embodiment, the guard parts 102d may be integrally formed with the second jaw 102. That is, the guard parts 102d may be formed to extend from side surfaces of the second jaw 102.

In an embodiment, the guard parts 102d may be formed as separate members and attached to the second jaw 102. For example, as shown in FIG. 5 or the like, a tissue guard 102c may be provided as a member including the guard parts 102d, and the tissue guard 102c may be coupled to the second jaw 102.

In other words, the tissue guard 102c may be a member including the guard parts 102d formed to face each other and may be formed overall in the shape of a hollow box with one surface (a lower surface) removed.

Meanwhile, the second jaw 102 may further include an anvil cover 102b. The anvil cover 102b may be formed corresponding to the shape of the second jaw 102 and may be coupled to an outer side surface (an upper surface) of the second jaw 102.

In addition, the tissue guard 102c may be coupled to the second jaw 102 by being attached to the outer side surface of the anvil cover 102b. In another embodiment, the tissue guard 102c may be formed integrally with the anvil cover 102b and coupled to the second jaw 102.

Referring to FIGS. 7 to 9 again, the guard parts 102d may be formed to extend toward the first jaw 101 such that at least one region thereof is positioned within the cartridge accommodation part 101a during the opening and closing motion of the first jaw 101 and the second jaw 102.

In other words, as the second jaw 102 rotationally moves with respect to the first jaw 101, causing a distance between the second jaw 102 and the first jaw 101 to decrease or increase, the guard part 102d may be positioned within the cartridge accommodation part 101a. Specifically, the guard parts 102d may each be configured to be positioned in a space between the cartridge 500 and the side wall part 101c when the cartridge 500 is accommodated in the cartridge accommodation part 101a.

In other words, the guard parts 102d may be disposed closer to the cartridge 500 than the side wall parts 101c.

That is, the side wall parts 101c of the first jaw 101 may each be positioned on an outer side of the guard part 102d. That is, the side wall parts 101c positioned on opposite sides of the first jaw 101 may be configured to cover outer side surfaces of the guard parts 102d formed on opposite sides of the second jaw 102.

In other words, the guard part 102d includes a first guard part and a second guard part, and the first guard part and the second guard part are disposed to face each other on opposite sides of the second jaw 102 with respect to a longitudinal axis of the second jaw 102. A width w2 between the first guard part and the second guard part is less than a width w1 between the side wall parts 101c.

Meanwhile, the end tool 100 further includes wires positioned within the cartridge accommodation part 101a in the longitudinal direction of the first jaw 101, and the guard parts 102d may be positioned between the cartridge 500 and the wires.

Specifically, the end tool 100 according to an embodiment of the present disclosure may include a plurality of forward-moving wires 301 and 302 and a backward-moving wire (not shown) for the movement of the operation member 140. The plurality of forward-moving wires 301 and 302 may be positioned within the cartridge accommodation part 101a of the first jaw 101, and may extend along opposite sides of the cartridge 500 from the proximal end 100p to the distal end 100d. That is, the forward-moving wires 301 and 302 may each be positioned between the side wall part 101c and the cartridge 500.

At this time, the guard parts 102d of the end tool 100 according to an embodiment of the present disclosure may be configured to be positioned between each of the forward-moving wires 301 and 302 and the cartridge 500.

In other words, the forward-moving wires 301 and 302 may be positioned between the guard parts 102d and the side wall parts 101c, respectively.

Meanwhile, in a state in which the first jaw 101 and the second jaw 102 are maximally spaced apart, at least one region of the guard parts 102d may be positioned within the cartridge accommodation part 101a of the first jaw 101.

Specifically, when the second jaw 102 is in a closed state, approaching the first jaw 101, the guard parts 102d may be maximally inserted into the cartridge accommodation part 101a.

Thus, in the closed state of the second jaw 102, the guard parts 102d may be restricted from coming into contact with the outside due to the first jaw 101.

On the other hand, when the second jaw 102 is in an open state, moving away from the first jaw 101, the guard parts 102d may be withdrawn from the cartridge accommodation part 101a.

At this time, at least some regions the guard parts 102d may be positioned within the cartridge accommodation part 101a. Specifically, at least one region of the guard parts 102d on the distal end 100d side may be inserted into the cartridge accommodation part 101a.

In other words, even in a state in which the first jaw 101 and the second jaw 102 are fully open, the lowermost portions of the guard parts 102d may still be positioned within the cartridge accommodation part 101a.

Meanwhile, in a closed state in which the second jaw 102 is rotated to be in closest proximity to the first jaw 101, a width of the first jaw 101 may define the maximum outer diameter of the end tool 100. That is, in the closed state of the end tool 100, the maximum outer diameter of the end tool 100 may be determined by the width of the first jaw 101. At this time, a width of the second jaw 102 may be less than the maximum outer diameter of the end tool 100. In other words, the width of the second jaw 102 may be less than the width of the first jaw 101.

In addition, the width of the first jaw 101 may be greater than a distance between the guard parts 102d in at least one region. In other words, the width between the guard parts 102d positioned on opposite sides of the second jaw 102 may be less than the width of the first jaw 101.

In addition, in the closed state in which the first jaw 101 and the second jaw 102 are rotated to be in closest proximity to each other, the maximum outer diameter of the end tool 100 may be defined as a maximum distance between the first jaw 101 and the second jaw 102 on a cross-sectional plane perpendicular to the longitudinal axis of the end tool 100. At this time, the guard parts 102d may be positioned in a space defined by the maximum outer diameter of the end tool 100.

That is, the width between the guard parts 102d may be less than the maximum outer diameter of the end tool 100.

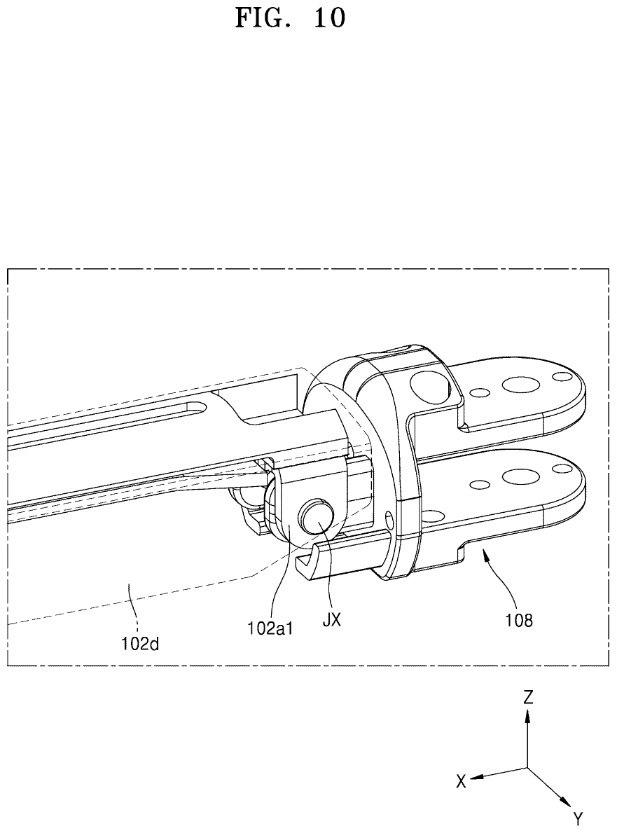

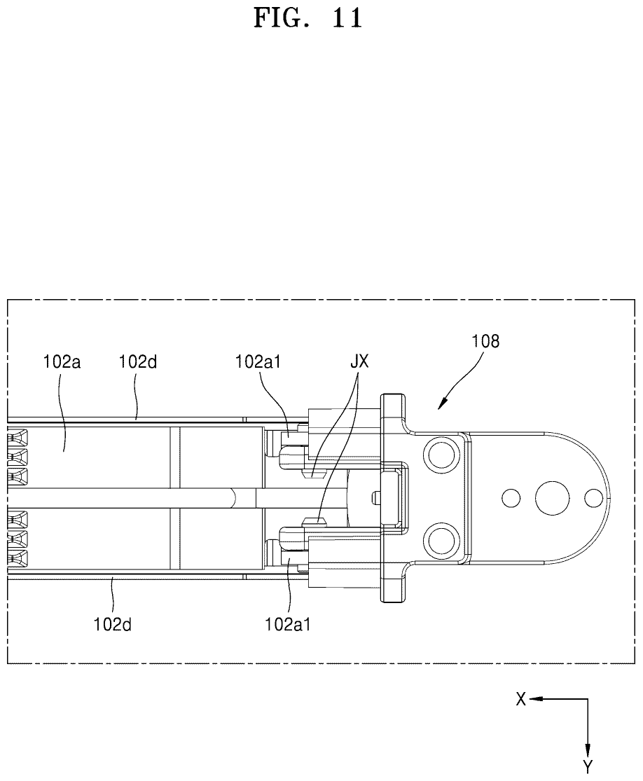

FIG. 10 is a view for describing the guard parts 102d and hinge parts 102a1 of the end tool 100 of FIG. 3. FIG. 11 is a bottom view illustrating the second jaw 102 of the end tool 100 of FIG. 10.

Referring to FIGS. 10 and 11, the second jaw 102 may include the hinge parts 102a1 including a first rotation shaft JX that serves as the center of rotational movement of the second jaw 102 with respect to the first jaw 101.

The hinge parts 102a1 may be formed on opposite sides of the second jaw 102 to extend from the second jaw 102 while being spaced apart from each other to face each other.

In addition, the first jaw 101 may include a coupling portion through which the first rotation shaft JX is inserted, and the hinge parts 102a1 of the second jaw 102 may be axially coupled to the coupling portion of the first jaw 101.

Here, the guard parts 102d according to an embodiment of the present disclosure may cover outer side portions of the hinge parts 102a1. In addition, the guard parts 102d may cover outer side portions of the first rotation shaft JX coupled to the hinge parts 102a1.

Thus, the guard parts 102d may serve to prevent the first rotation shaft JX, which is coupled to the hinge parts 102a1, from detaching.

Accordingly, separate processes such as laser welding to ensure the rotation shaft does not detach from the hinge part 102a1 during the assembly of the first jaw 101 and the second jaw 102 may be eliminated.

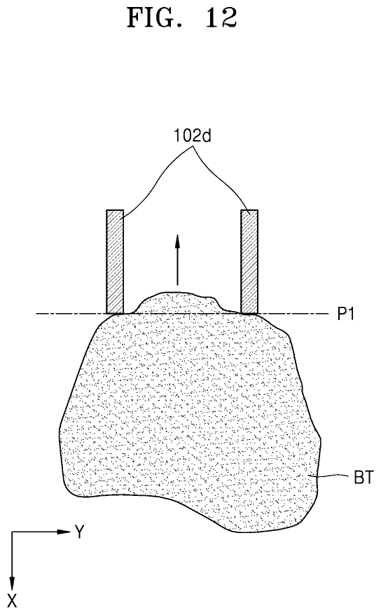

FIG. 12 is a view for describing functions of the guard parts 102d of the end tool 100 according to an embodiment of the present disclosure.

The guard parts 102d of the end tool 100 according to an embodiment of the present disclosure may restrict the insertion of body tissue into a preset region when the body tissue is clamped between the first jaw 101 and the second jaw 102.

Specifically, the insertion of body tissue into the space on the proximal end 100p side beyond the position in which the guard parts 102d are formed may be restricted.

For example, staple holes through which a plurality of staples may be ejected to the outside may be formed in a cover of the cartridge 500, but such staple holes may not be formed in the cartridge 500 on the proximal end 100p side. In other words, a region in which the staples are not ejected may be present in the cartridge 500 on the proximal end 100p side.

Accordingly, during the stapling motion with body tissue interposed between the first jaw 101 and the second jaw 102, when the body tissue is inserted up to the proximal end 100p side of the cartridge 500, a portion of the body tissue may not be stapled.

The guard part 102d may prevent the above-described situation by restricting the insertion of body tissue into a predefined region.

FIG. 12 is a view schematically illustrating the situation in which the guard parts 102d come into contact with body tissue and is a top view of the guard parts 102d and the body tissue.

Meanwhile, referring to FIG. 12, since body tissue BT, which is the target of the stapling procedure, is generally not rigid, the body tissue BT may protrude convexly between the guard parts 102d when interposed between the first jaw 101 and the second jaw 102 and comes into contact with the guard parts 102d.

For example, as shown in FIG. 12, an imaginary plane P1 connecting two contact points at which the guard parts 102d are in contact with the body tissue BT may be regarded as a supporting line that prevents the body tissue BT from being inserted beyond a predefined region. However, as the distance between the guard parts 102d increases, the amount of body tissue protruding inward beyond the supporting line may also increase.

Here, the guard parts 102d of the end tool 100 according to an embodiment of the present disclosure may be formed such that the width w2 between the guard parts 102d is less than the width w1 between the side wall parts 101c. Accordingly, as the distance between the guard parts 102d decreases, the amount of protruding body tissue may be reduced, which can contribute to the stability of the procedure.

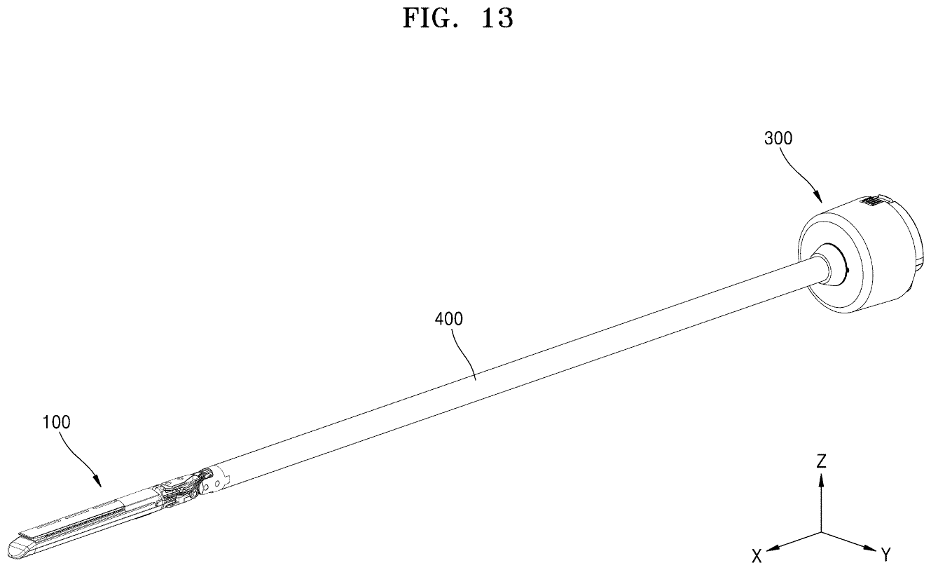

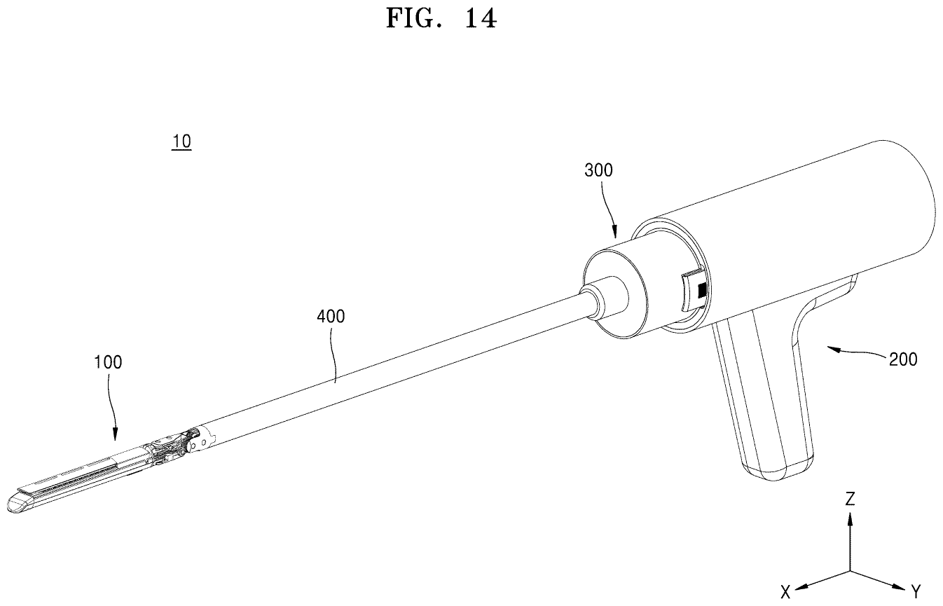

FIG. 13 is a perspective view illustrating a reload assembly to which the end tool according to an embodiment of the present disclosure is applied, and FIG. 14 is a perspective view illustrating a surgical instrument to which the end tool according to an embodiment of the present disclosure is applied.

A surgical instrument 10 may include the end tool 100, a manipulation part 200, a connection part 400, and a power transmission part 300.

Meanwhile, as illustrated in FIG. 13, a configuration including the end tool 100, the connection part (or shaft) 400, and the power transmission part 300 will be distinguished and referred to as the reload assembly.

Here, the connection part 400 is formed in the shape of a hollow shaft, and one or more wires and electric wires may be accommodated therein. For convenience of description of the present embodiment, the shaft will be referred to and described as the connection part 400. The manipulation part 200 is coupled to one end portion of the connection part 400, the end tool 100 is coupled to another end portion thereof, and the connection part 400 may serve to connect the manipulation part 200 to the end tool 100. As an example, the connection part 400 may include a straight part, and although not shown in the drawings, the connection part 400 may include one or more curved parts to increase ease of use and control the arrangement of components for manipulation.

The power transmission part 300 may be formed on another end portion of the connection part 400 and may serve to transmit power generated from a power generation part to be described later to the end tool 100. For example, the power transmission part 300 may be positioned between the end tool 100 and the manipulation part 200. As will be described later, when a user such as a medical doctor manipulates the manipulation part 200, the power generation part generates power to control the end tool 100, and the generated power may be transmitted to the end tool 100 through the power transmission part 300. The power transmission part 300 may include a plurality of wires, a plurality of pulleys, a plurality of links, a plurality of joints, a plurality of gears, and the like.

A user may operate the end tool 100 by manipulating the manipulation part 200. For example, the manipulation part 200 is a configuration for the user to input signals to control the motions of the end tool 100. That is, the manipulation part 200 may be said to be a configuration that receives signals from the user to control the operation of the end tool 100. Here, the signals for controlling the motions of the end tool 100 may correspond to mechanical manipulations such as pressing a button or switch, or rotating or moving a particular member, and may also be electrical signals generated by such mechanical manipulations, but the present disclosure is not limited thereto. The manipulation part 200 is provided as an interface to be directly controlled by a medical doctor, for example, provided in a gun shape, a tongs shape, a stick shape, a lever shape, or the like, and when the medical doctor controls the manipulation part 200, the end tool 100, which is connected to the corresponding interface and inserted into the body of a surgical patient, performs a certain motion, thereby performing surgery. Here, the manipulation part 200 is illustrated in FIG. 14 as being formed in a gun shape, but the concept of the present disclosure is not limited thereto, and various types of manipulation parts that can be connected to the end tool 100 and manipulate the end tool 100 may be possible.

The manipulation part 200 may include a housing forming the exterior of the manipulation part 200. As will be described later, at least a portion of the power generation part configured to generate power to control the end tool 100 may be accommodated inside the housing. In addition, a circuit unit for controlling the operation of the power generation part and slip rings for supplying electrical energy to the power generation part, for connecting communication, transmitting various other signals may be accommodated inside the housing.

A handle may be formed on the manipulation part 200. The handle is a part for a user to grip. Thus, the user can use the surgical instrument 10 according to the present disclosure while gripping the handle of the manipulation part 200.

Meanwhile, although not shown in the drawings, a button, a switch, a lever, and the like for controlling various motions of the end tool 100 may be further formed in the manipulation part.

Hereinafter, an end tool and a surgical instrument according to another embodiment of the present disclosure will be described in detail.

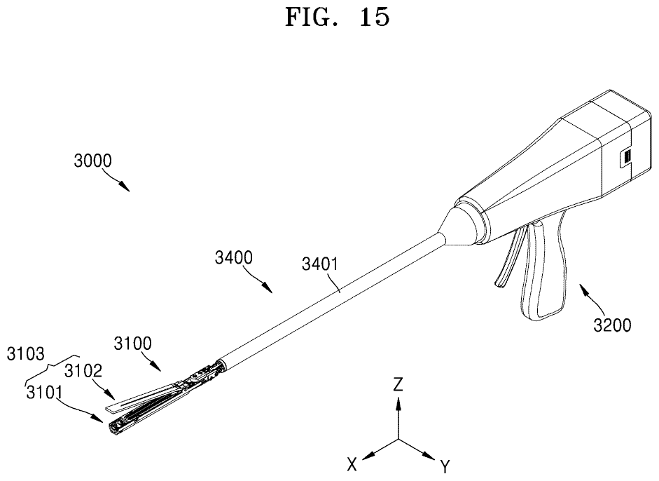

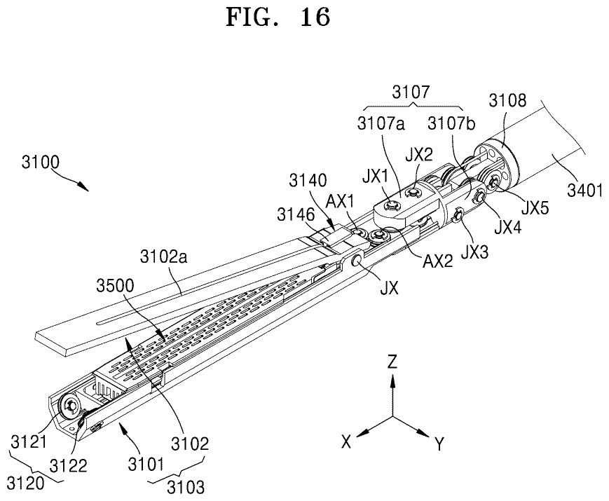

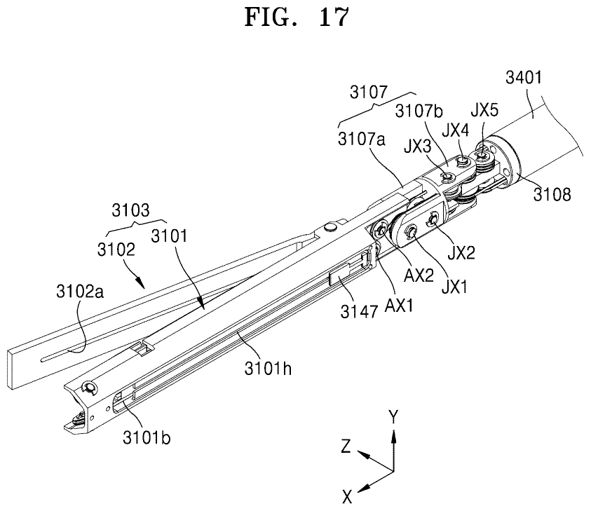

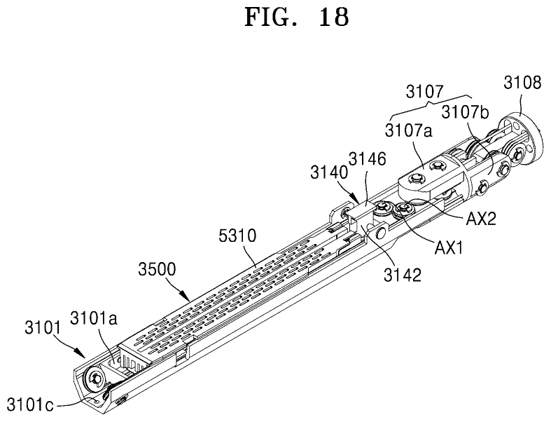

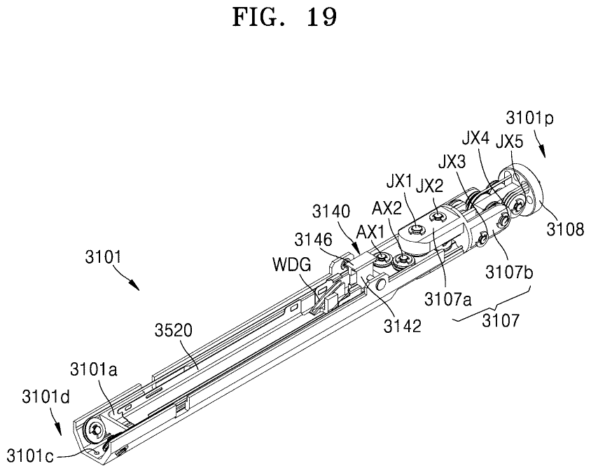

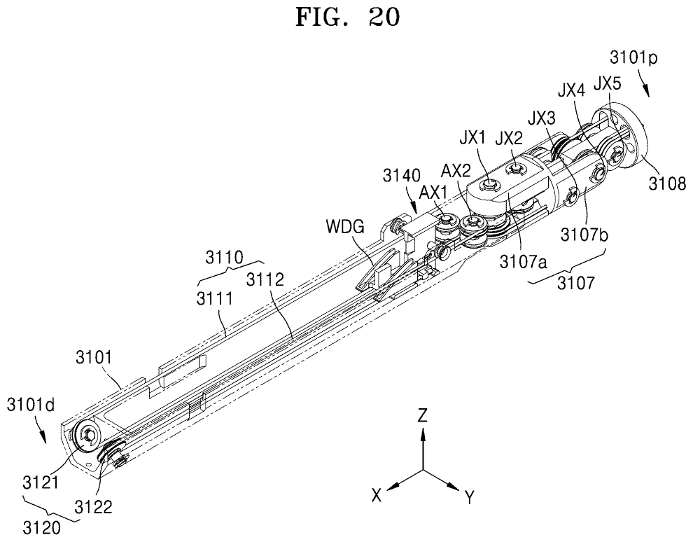

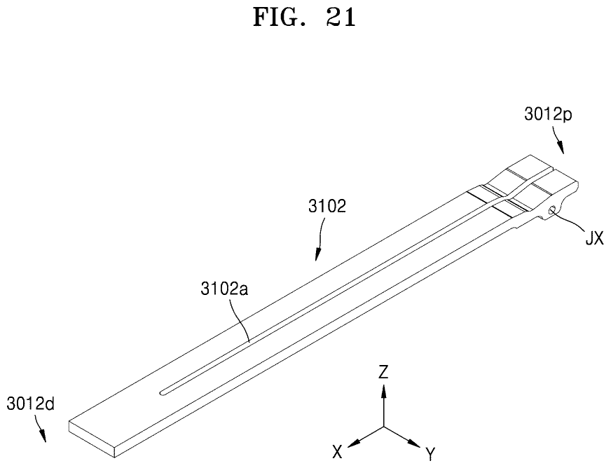

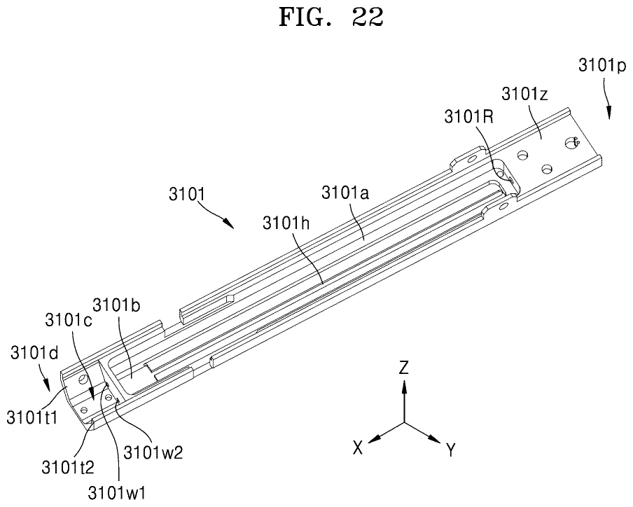

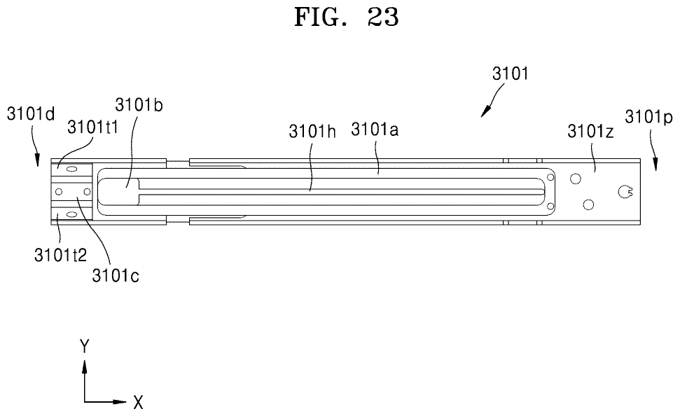

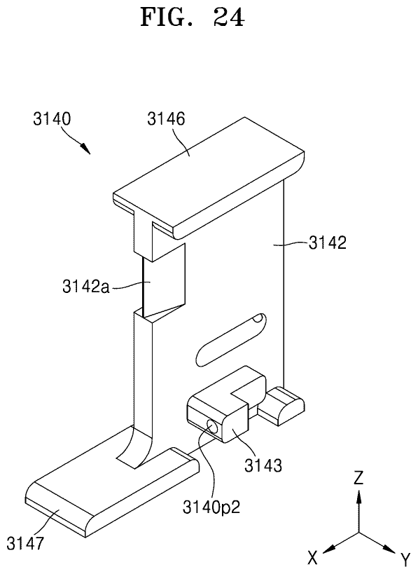

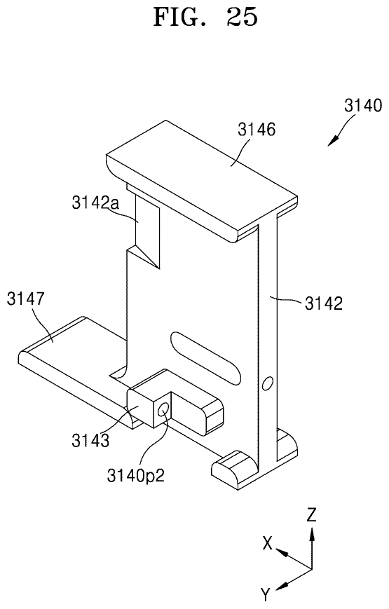

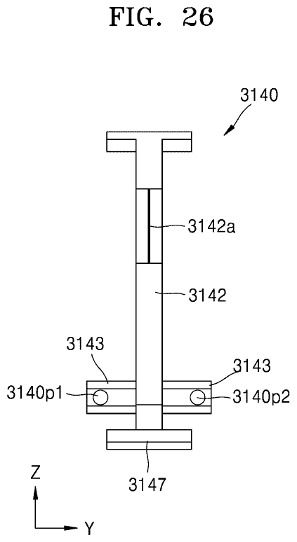

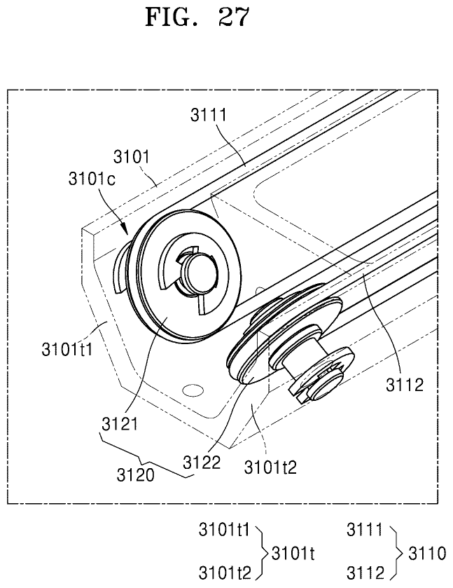

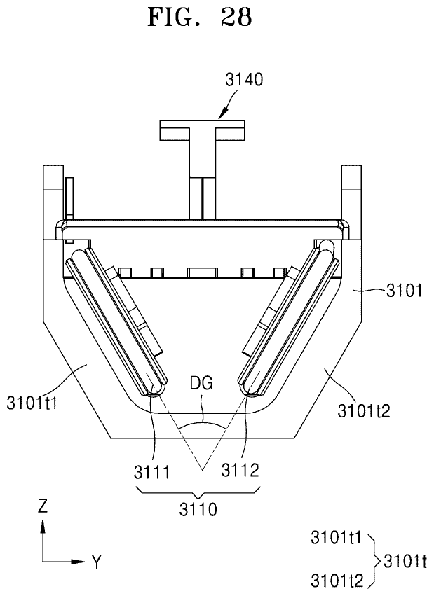

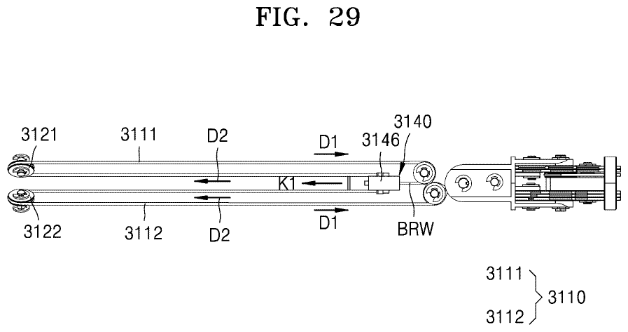

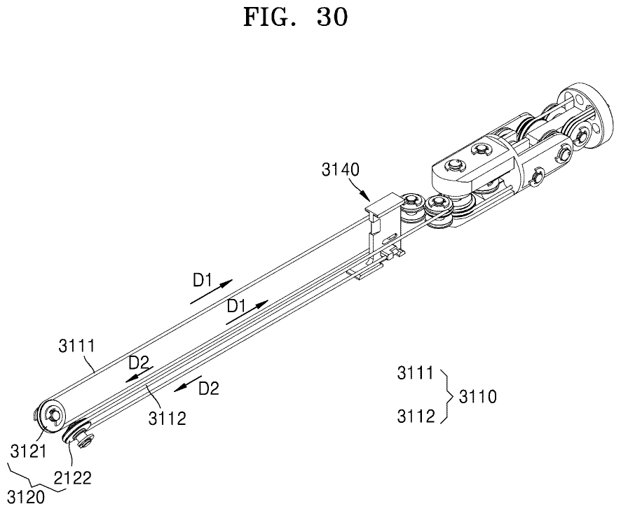

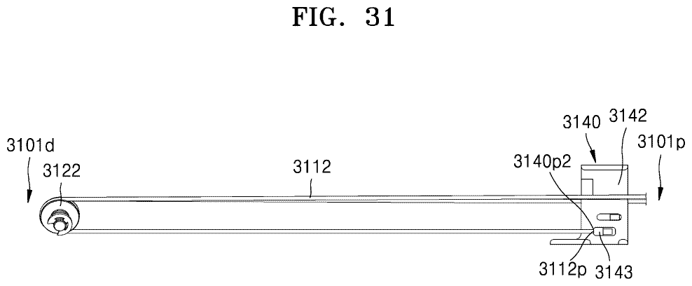

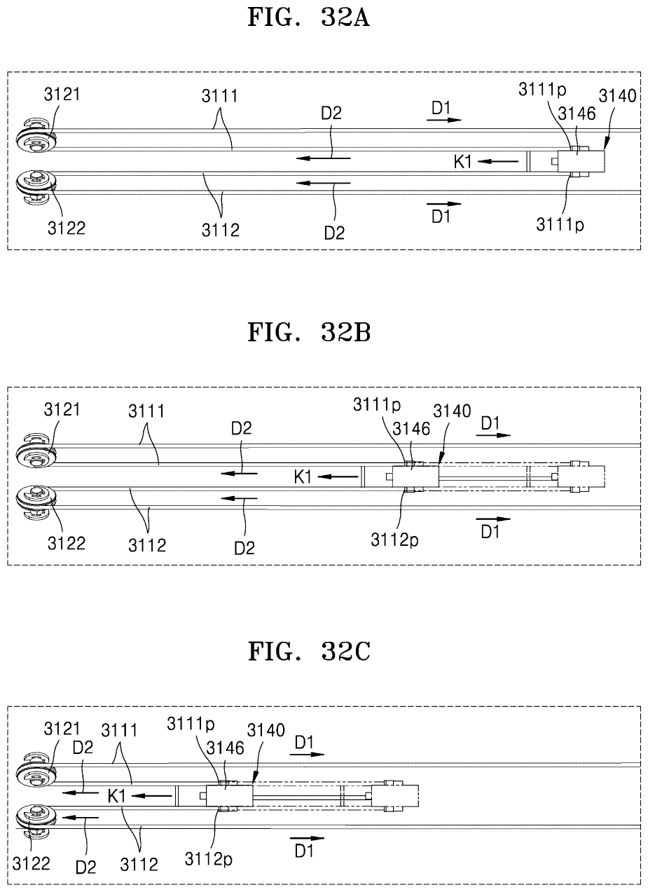

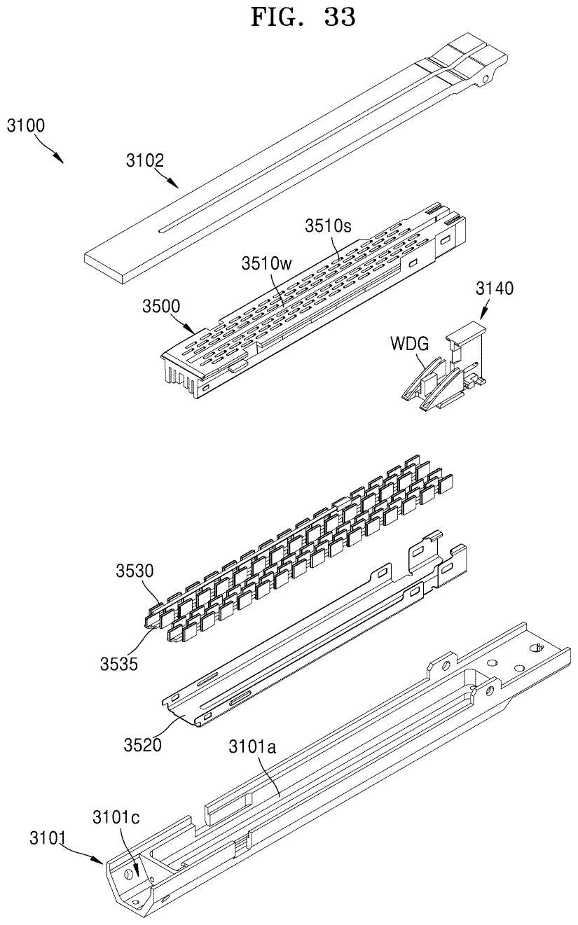

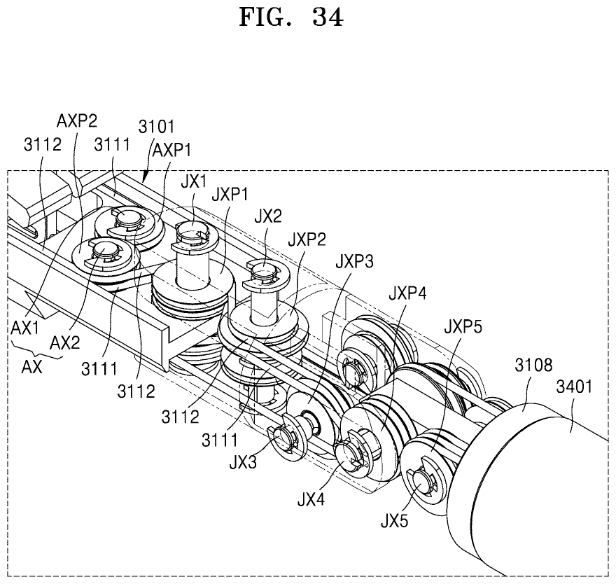

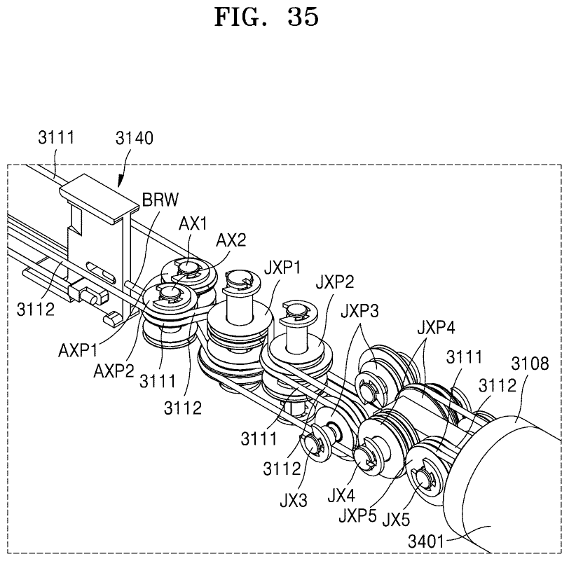

FIG. 15 is a schematic perspective view of a surgical instrument according to another embodiment of the present disclosure. FIG. 16 is a schematic perspective view for describing an end tool illustrated in FIG. 15. FIG. 17 is a perspective view of the end tool of FIG. 16 viewed from another direction. FIG. 18 is a schematic perspective view of the end tool of FIG. 16 with a second jaw removed. FIG. 19 is a schematic perspective view of the end tool of FIG. 18 with a cartridge removed. FIG. 20 is a transparent perspective view of FIG. 19. FIG. 21 is a perspective view schematically illustrating the second jaw of the end tool of FIG. 16. FIG. 22 is a perspective view schematically illustrating a first jaw of the end tool of FIG. 16. FIG. 23 is a plan view schematically illustrating the first jaw of the end tool of FIG. 16. FIG. 24 is a perspective view illustrating an operation member of the end tool of FIG. 16. FIG. 25 is a perspective view of the operation member of FIG. 24 viewed from another direction. FIG. 26 is a front view of the operation member of FIG. 24 viewed in one direction. FIG. 27 is a schematic perspective view illustrating a portion of the end tool of FIG. 16. FIG. 28 is a front view of FIG. 27 viewed in one direction. FIG. 29 is a schematic plan view for describing the operation member, a fixed pulley, and a forward-moving wire of the end tool of FIG. 16. FIG. 30 is a schematic perspective view for describing the operation member, the fixed pulley, and the forward-moving wire of the end tool of FIG. 16. FIG. 31 and FIGS. 32A to 32C are schematic views for describing an operation of the operation member of the end tool of FIG. 16. FIG. 33 is a perspective view illustrating the first jaw and the cartridge of the surgical instrument of FIG. 15. FIGS. 34 and 35 are views for describing a switching pulley, a yaw pulley, and a pitch pulley of the end tool of the surgical instrument of FIG. 15.

A surgical instrument 3000 according to the present embodiment may include an end tool 3100, a manipulation part 3200, and a connection part 3400.

Hereinafter, the end tool 3100 of the surgical instrument 3000 of FIG. 15 will be described in more detail.

The end tool 3100 may include a jaw 3103, a plurality of fixed pulleys 3120, and a plurality of forward-moving wires 3110. The plurality of fixed pulleys 3120 may include two or more pulleys, for example, a first fixed pulley 3121 and a second fixed pulley 3122. The plurality of forward-moving wires 3110 include two or more wires, and may include, for example, a first forward-moving wire 3111 and a second forward-moving wire 3112.

The jaw 3103 may perform various functions, for example, a grip motion, and may include a pair of jaws, e.g., a first jaw 3101 and a second jaw 3102 as a specific example. Here, each of the first jaw 3101 and the second jaw 3102, or a component encompassing the first jaw 3101 and the second jaw 3102 may be referred to as the jaw 3103.

The first jaw 3101 and the second jaw 3102 may be positioned to face each other, may move closer to or move away from each other, and may be configured to rotationally move around, for example, one shaft JX.

A cartridge 3500 may be positioned to be accommodated in the first jaw 3101, and a plurality of staples are positioned inside the cartridge 3500. When an operation member 3140 receives a force through the forward-moving wires 3110 while the first jaw 3101 and the second jaw 3102 are close to each other, such as when the first jaw 3101 and the second jaw 3102 are closed with body tissue interposed therebetween, the operation member 3140 may push and raise the staples while moving toward a distal end 3101d of the first jaw 3101, thereby performing stapling. At this point, one or more clamps 3146 and 3147 of the operation member 3140 may protrude to the outside of the first jaw 3101 and the second jaw 3102, allowing the operation member 3140 to move forward while applying pressure to an outer surface of the first jaw 3101 and the second jaw 3102, which facilitates the smooth progression of a stapling process. In an optional embodiment, the cartridge 3500 may include a case 3520 corresponding to the bottom, and the case 3520 is positioned in the first jaw 3101.

Meanwhile, the operation member 3140 may be used together with a wedge WDG. For example, the wedge WDG may be prepared separately from the operation member 3140 and then disposed adjacent to the operation member 3140 in the first jaw 3101. In addition, as another example, the operation member 3140 and the wedge WDG may be integrally formed. The wedge WDG may be positioned on at least one side of a body 3142 of the operation member 3140 and may be formed to have a predetermined inclined surface. That is, the wedge WDG may be formed to be inclined by a certain degree with respect to an extension direction of the end tool 3100. In other words, the wedge WDG may be formed to have a greater height at a proximal end 3101p side of the first jaw 3101 than a distal end 3101d side of the first jaw 3101.

The wedge WDG may be configured to be sequentially in contact with withdrawal members 3535 (refer to FIG. 33) or a plurality of staples 3530 (refer to FIG. 33) positioned in the cartridge 3500, and may serve to sequentially push and raise the staples 3530.

The plurality of fixed pulleys 3120 may be positioned in the first jaw 3101 to be closer to the front of the cartridge 3500, i.e., to the distal end 3101d of the first jaw 3101, than the cartridge 3500. For example, a plurality of fixed pulleys 3120 may be positioned in a front space 3101c of the first jaw 3101, and details thereof will be described below.

In addition, the end tool 3100 of the surgical instrument of the present embodiment may include one or more members, such as joint members, that connect the jaw 3103 to the connection part 3400. Further, in an optional embodiment, the end tool 3100 may include an end tool hub 3108 and a pitch hub 3107.

The end tool hub 3108 may be disposed to connect the end tool 3100 to the straight part 3401 of the connection part 3400.

As an example, the end tool hub 3108 may have a pulley shaft JX4 corresponding thereto, and the pulley shaft JX4 may be a pitch rotation shaft. As a specific example, the end tool 3100 may perform a vertical rotational motion around the pulley shaft JX4 based on the drawing. In addition, one or more pulleys may be disposed to be adjacent to the pulley shaft JX4.

The end tool hub 3108 may be in the form of a bar extending from the center of a surface thereof that corresponds to the connection part 3400, i.e., a bar extending from the center of a disk-shaped main region. The pulley shaft JX4 and a pulley shaft JX5 different from the pulley shaft JX4 may further correspond to a region of the bar.

The pitch hub 3107 is connected to the end tool hub 3108 and the jaw 3103. The pitch hub 3107 may be axially coupled to the end tool hub 3108 with respect to one pulley shaft, i.e., the pulley shaft JX4. The pitch hub 3107 may rotationally move around one pulley shaft, i.e., the pulley shaft JX4 while connected to the end tool hub 3108. That is, the end tool 3100 may perform a pitch motion as the pitch hub 3107 rotates around one pulley shaft, i.e., the pulley shaft JX4 with respect to the end tool hub 3108.

Further, the jaw 3103 of the end tool 3100 may be axially coupled to the pitch hub 3107 with respect to one pulley shaft, i.e., a pulley shaft JX1. The jaw 3103 may rotate around one pulley shaft, i.e., the pulley shaft JX1 while connected to the pitch hub 3107. That is, the jaw 3103 of the end tool 3100 may rotate around one pulley shaft, i.e., the pulley shaft JX1 with respect to the pitch hub 3107, thereby performing a yaw motion.

As a result, the yaw motion of the end tool 3100 includes a rotational motion of the jaw 3103 around one pulley shaft, i.e., the pulley shaft JX1 with respect to the pitch hub 3107, and the pitch motion of the end tool 3100 includes a rotational motion of the jaw 3103 coupled to the pitch hub 3107, which occurs as the pitch hub 3107 rotates around one pulley shaft, i.e., the pulley shaft JX4 together with the end tool hub 3108.

The pitch hub 3107 may include a first hub 3107a and a second hub 3107b.

The first hub 3107a of the pitch hub 3107 may be connected to the jaw 3103. As an example, the first hub 3107a may be elongated to connect to one region of the first jaw 3101, and specifically, may have two bars that are formed side by side to face each other and coupled to each other by placing one region of the first jaw 3101 therebetween.

The second hub 3107b of the pitch hub 3107 may be connected to the end tool hub 3108, for example, may have two bars that are formed side by side to face each other, and may be coupled to each other by placing one region of the end tool hub 3108 therebetween.

As described above, the pulley shaft JX5 different from one pulley shaft, i.e., the pulley shaft JX4 may be positioned in the end tool hub 3108 to be spaced apart from the pulley shaft JX4 and closer to the connection part 3400 (refer to FIG. 15) than the pulley shaft JX4. The pulley shaft JX4 and the pulley shaft JX5 may have axes in directions parallel to each other.

A pulley shaft JX2, which is different from the pulley shaft JX1, is disposed in the pitch hub 3107 in a direction adjacent to and parallel to the pulley shaft JX1. In addition, a pulley shaft JX3 and the pulley shaft JX4 may be formed in a direction different from (for example, intersecting or orthogonal to) the direction in which the pulley shaft JX1 and the pulley shaft JX2 are disposed, and may be sequentially positioned in a direction toward (or away from the operation member) the connection part 3400.

The pulley shaft JX4 may be a pitch motion shaft of the end tool 3100, and the pulley shaft JX1 may be a yaw motion shaft.

The pulley shaft JX3 and the pulley shaft JX5 may be pitch auxiliary pulley shafts, and the pulley shaft JX2 may be a yaw auxiliary pulley shaft. One or more driving wires, such as a wire configured to transmit a driving force for a pitch motion or a yaw motion may have at least one region in contact with or wound around the pulley shafts JX1, JX2, JX3, JX4, and JX5.

The pulley shafts JX2, JX3, and JX5 adjacent to the pulley shaft JX4, which is a pitch motion shaft, and the pulley shaft JX1, which is a yaw motion shaft, may control paths along which the driving wires are wound around the pulley shaft JX4 and the pulley shaft JX1 to secure the efficiency of the arrangement of the driving wires and stabilize the paths for transmitting forces through the driving wires.

In addition, at least one region of the forward-moving wire 3110 may be in contact with or wound around the pulley shafts JX1, JX2, JX3, JX4, and JX5.

A more detailed description of the arrangement of the pulley shafts JX1, JX2, JX3, JX4, and JX5 will be provided below.

In the end tool 3100, one or more switching pulley shafts AX1 and AX2 may be positioned, and one or more pulleys corresponding to the switching pulley shafts AX1 and AX2 may be disposed.

For example, the switching pulley shafts AX1 and AX2 may be positioned in the jaw 3103, specifically, at the proximal end 3101p side of the first jaw 3101, and may be disposed closer to the distal end 3101d of the first jaw 3101 than at least the above-described pulley shafts JX1, JX2, JX3, JX4, and JX5.

The switching pulley shafts AX1 and AX2 may be shafts formed in parallel to each other, and may be disposed to have different backward and forward positions with respect to each other such that the switching pulley shaft AX1 and the switching pulley shaft AX2 are sequentially disposed with respect to the distal end 3101d of the first jaw 3101 and some regions of the switching pulley shaft AX1 and the switching pulley shaft AX2 are overlap each other.

The switching pulley shafts AX1 and AX2 may be regions where at least one region of the forward-moving wires 3110 is wound or comes into contact to organize and guide the path of the forward-moving wire 3110 before entering the pulley shafts JX1, JX2, JX3, JX4, and JX5. A more detailed description of the arrangement of the switching pulley shafts AX1 and AX2 will be provided below.

As shown in FIG. 20, the first forward-moving wire 3111 and the second forward-moving wire 3112 may be correspondingly wound around the first fixed pulley 3121 and the second fixed pulley 3122 in the first jaw 3101 to be redirected, and connected to the rear of the end tool 3100 via at least one region of each of the switching pulley shafts AX1 and AX2 and the pulley shafts JX1, JX2, JX3, JX4, and JX5. Furthermore, the first forward-moving wire 3111 and the second forward-moving wire 3112 may further extend to the manipulation part 3200 (refer to FIG. 15) via the connection part 3400 to be precisely controlled. Accordingly, precise motion control of the operation member 3140 may be easily implemented, and details thereof will be described below.

The jaw 3103 of the end tool 3100 will be described in more detail.

FIG. 21 is a perspective view schematically illustrating the second jaw of the end tool of FIG. 16.

The second jaw 3102 may be formed in an elongated bar shape as a whole, and for example, the second jaw 3102 may be formed in a rod shape to correspond to the first jaw 3101 in at least one region.

A proximal end 3102p of the second jaw 3102 may include a region that is coupled to the first jaw 3101. As an example, the proximal end 3102p is configured to be rotatable around a rotation shaft JX of the proximal end 3102p with respect to the first jaw 3101

The second jaw 3102 may have various forms, and as a specific example, a plurality of anvil grooves may be formed in at least one region of a surface of the second jaw 3102 facing the first jaw 3101, and the anvil groove may have a shape corresponding to the shape of the staple 3530.

The anvil groove of the second jaw 3102 may serve as a support for allowing the staple 3530 to be bent when the operation member 3140 push and raise the staple 3530 during a staple motion.

The second jaw 3102 includes a guide groove 3102a. The guide groove 3102a may have a shape elongated in a longitudinal direction of the second jaw 3102.

The guide groove 3102a may be configured to guide the operation member 3140, and may have a through-hole shape in a region facing the operation member 3140. Through this, one region of the operation member 3140, such as at least one region of the body 3142 of the operation member 3140, or a first clamp 3146 connected thereto may pass through the guide groove 3102a to exit to the outside of the second jaw 3102. When the operation member 3140 moves forward, the first clamp 3146 may pass through the guide groove 3102a of the second jaw 3102 to be exposed to the outside of the second jaw 3102, and may come into contact with an upper surface of the second jaw 3102 or apply pressure thereto. As the operation member 3140 moves, the first clamp 3146 applies pressure on the upper surface of the second jaw 3102 and a second clamp 3147 to be described below applies pressure on a lower surface of the first jaw 3101 such that a gap between the second jaw 3102 and the first jaw 3101 decreases, allowing the second jaw 3102 to naturally remain in a closed state with respect to the first jaw 3101.

FIG. 22 is a perspective view schematically illustrating the first jaw of the end tool of FIG. 16. FIG. 23 is a plan view schematically illustrating the first jaw of the end tool of FIG. 16.

Referring to FIGS. 22 and 23 and the like, the first jaw 3101 is formed in an elongated bar shape as a whole, and a rotation shaft may be positioned in the proximal end such that the first jaw 3101 is rotationally movable, and such a rotation shaft may correspond to the rotation shaft JX formed in the second jaw 3102 described above. In addition, the cartridge 3500 (refer to FIG. 16) may be accommodated closer to the distal end 3101d side than the rotation shaft.

For example, the first jaw 3101 may be formed entirely in the form of a hollow box with one surface (upper surface) thereof is removed, such that a cartridge accommodation part 3101a capable of accommodating the cartridge 3500 may be formed inside the first jaw 3101. That is, the first jaw 3101 may be formed in a substantially “U” shape in cross section.

A guide groove 3101h may be formed in a bottom surface of the first jaw 3101, the bottom surface opposite to an upper open region formed by removing one surface. Specifically, the guide groove 3101h may be configured to guide a linear motion of the operation member 3140.

The guide groove 3101h may be configured to guide the operation member 3140, and may have a through-hole shape in a region facing the operation member 3140. Through this, one region of the operation member 3140, such as at least one region of the body 3142 of the operation member 3140, or the second clamp 3147 connected thereto may pass through the guide groove 3101h to exit to the outside of the first jaw 3101. When the operation member 3140 moves forward, the second clamp 3147 may pass through the guide groove 3101h of the first jaw 3101 to be exposed to the outside of the first jaw 3101, and may come into contact with the lower surface of the first jaw 3101 or apply pressure thereto. As the operation member 3140 moves, the second clamp 3147 applies pressure on the lower surface of the first jaw 3101 and the first clamp 3146 applies pressure on the upper surface of the second jaw 3102 such that that gap between the second jaw 3102 and the first jaw 3101 decreases, allowing the second jaw 3102 to naturally remain in a closed state with respect to the first jaw 3101.

In an optional embodiment, the first jaw 3101 may include a window 3101b. After operating the operation member 3140 or using the end tool 3100, the second clamp 3147 of the operation member 3140 may be positioned corresponding to the window 3101b, and the coupled state of the first jaw 3101 and the operation member 3140 may be released.

The first jaw 3101 may include the front space 3101c positioned ahead of the cartridge accommodation part 3101a.

For example, the front space 3101c may be disposed closer to the distal end 3101d of the first jaw 3101 than the cartridge accommodation part 3101a. The plurality of fixed pulleys 3120 may be positioned in the front space 3101c, for example, the first fixed pulley 3121 and the second fixed pulley 3122 may be positioned in the front space 3101c (refer to, for example, FIGS. 16 to 20).

Two outer side surfaces of the front space 3101c include a first side surface 3101t1 and a second side surface 3101t2, and the first fixed pulley 3121 and the second fixed pulley 3122 may be disposed to correspond to the first side surface 3101t1 and the second side surface 3101t2, respectively.

Each of the first side surface 3101t1 and the second side surface 3101t2 may be formed to have an inclined shape. For example, the first side surface 3101t1 and the second side surface 3101t2 may be shaped such that a gap therebetween decreases toward downward, rather than being parallel to each other with a uniform gap. As a specific example, the gap between the first side surface 3101t1 and the second side surface 3101t2 may be configured to be smaller in a direction away from the second jaw 3102.

Since the first fixed pulley 3121 and the second fixed pulley 3122 are disposed to correspond to the first side surface 3101t1 and the second side surface 3101t2, respectively, the first fixed pulley 3121 and the second fixed pulley 3122 may be disposed to have an inclined shape such that a gap therebetween decreases in a direction away from the second jaw 3102 (refer to FIG. 16). The first fixed pulley 3121 and the second fixed pulley 3122 may be symmetrically shaped and may have the same size as each other.

In addition, a first path 3101w1 and a second path 3101w2 may be formed adjacent to the front space 3101c. For example, the first path 3101w1 and the second path 3101w2 may have the form of through holes formed in a barrier wall, and may be regions through which the first forward-moving wire 3111 and the second forward-moving wire 3112 pass, respectively.

In an optional embodiment, when a backward-moving wire is placed, the first jaw 3101 may include a rear side path 3101R corresponding to the backward-moving wire.

In addition, the first jaw 3101 may include a coupling region 3101z in a region adjacent to the proximal end 3101p. The coupling region 3101z is a region coupled to the pitch hub 3107 and may be in the form of a plate elongated to correspond to, for example, the first hub 3107a of the pitch hub 3107. The coupling region 3101z may be positioned and coupled between two bars formed on the first hub 3107a of the pitch hub 3107.

An operation member 3140 will now be described in detail.

FIG. 24 is a perspective view illustrating the operation member of the end tool of FIG. 16. FIG. 25 is a perspective view of the operation member of FIG. 24 viewed from another direction. FIG. 26 is a front view of the operation member of FIG. 24 viewed in one direction.

The operation member 3140 may include the body 3142, the first clamp 3146, and the second clamp 3147. Meanwhile, the operation member 3140 may be used together with the wedge WDG (refer to FIGS. 19 and 20). For example, the wedge WDG may be prepared separately from the operation member 3140 and then disposed adjacent to the operation member 3140 in the first jaw 3101. In addition, as another example, the operation member 3140 and the wedge WDG may be integrally formed as one body. In the present specification, for convenience of description, the operation member 3140 and the wedge WDG will be described and illustrated in the drawings with the assumption that the operation member 3140 and the wedge WDG are prepared separately.

The wedge WDG may be positioned on at least one side of the body 3142 and may be formed to have a predetermined inclined surface. That is, the wedge WDG may be formed to be inclined by a certain degree with respect to the extension direction of the end tool 3100. In other words, the wedge WDG may be formed to have a greater height at the proximal end 3101p side of the first jaw 3101 than the distal end 3101d side of the first jaw 3101.

The wedge WDG may be configured to be sequentially in contact with the withdrawal members 3535 (refer to FIG. 33) or the plurality of staples 3530 (refer to FIG. 33) and may serve to sequentially push and raise the staples 3530.

The body 3142 may be in the form of an elongated column, such as a plate-shaped column. In addition, a blade region 3142a may be formed in one region of the body 3142, and an edge sharply formed to cut tissue may be formed in the blade region 3142a. The tissue positioned between the first jaw 3101 and the second jaw 3102 may be cut as at least a portion of the edge positioned in the blade region 3142a of the body 3142 is withdrawn to the outside of the first jaw 3101 and the cartridge 3500.

The first clamp 3146 may be formed in one region of the body 3142, and the second clamp 3147 may be formed in another region different from the one region. For example, the body 3142 may be positioned between the first clamp 3146 and the second clamp 3147.

The first clamp 3146 and the second clamp 3147 may be formed to have a region with a width at least greater than that of the body 3142. Accordingly, the first clamp 3146 may be inserted into and pass through the guide groove 3102a formed in the second jaw 3102 in the longitudinal direction to be positioned or brought into contact with the upper surface of the second jaw 3102 and, at the same time, the second clamp 3147 may be inserted into and pass through the guide groove 3101h formed in the first jaw 3101 in the longitudinal direction to be positioned or brought into contact with the lower surface of the first jaw 3101, so that the first clamp 3146 and the second clamp 3147 may move. Thus, when the operation member 3140 moves, the first clamp 3146 and the second clamp 3147 may apply forces in directions that bring the second jaw 3102 and the first jaw 3101 closer to each other.

As a result, when the operation member 3140 moves from the proximal end 3101p of the first jaw 3101 toward the distal end 3101d of the first jaw 3101, a motion of decreasing a distance between the second jaw 3102 and the first jaw 3101, i.e., a closing motion of the jaw 3103, may be naturally implemented through the first clamp 3146 and the second clamp 3147.

The first clamp 3146 and the second clamp 3147 may be located at different positions with respect to a forward-facing direction of the body 3142. For example, the second clamp 3147 may be positioned further forward than the first clamp 3146, for example, the second clamp 3147 may be disposed closer to the distal end 3101d of the first jaw 3101 than the first clamp 3146 when the operation member 3140 is positioned in the first jaw 3101. Accordingly, the operation member 3140 may move forward while the first jaw 3101 and second jaw 3102 are in the closed state, so that the first jaw 3101 and second jaw 3102 can be maintained with greater efficiency and stability while performing stapling.

A first connection region 3140p1 and a second connection region 3140p2 may be formed in one region of the body 3142, such as one region in a front side of the body 3142, specifically, a region of the body 3142 facing the distal end 3101d of the first jaw 3101.

The first connection region 3140p1 and the second connection region 3140p2 may be regions to which the first forward-moving wire 3111 and the second forward-moving wire 3112 are connected, respectively, and may each be in the form of a fixing groove such that respective one end portion region of the first forward-moving wire 3111 and the second forward-moving wire 3112 is accommodated or fixed thereto. When the first forward-moving wire 3111 and the second forward-moving wire 3112 are connected to the first connection region 3140p1 and the second connection region 3140p2 to pull the first forward-moving wire 3111 and the second forward-moving wire 3112, respectively, forces pulling the first forward-moving wire 3111 and the second forward-moving wire 3112 are transmitted to the operation member 3140 through the first connection region 3140p1 and the second connection region 3140p2 so that the operation member 3140 may move, i.e., move forward.

In an optional embodiment, the first connection region 3140p1 and the second connection region 3140p2 may be formed on side portions 3143 of the operation member 3140, respectively.

The side portions 3143 may be formed to protrude outward from both side surfaces of the body 3142, respectively. By forming the first connection region 3140p1 and the second connection region 3140p2 on the side portions 3143 formed to protrude from opposite sides of the body 3142, spaces for respectively connecting the first forward-moving wire 3111 and the second forward-moving wire 3112 to the first connection region 3140p1 and the second connection region 3140p2 can be easily secured.

Further, by forming the first connection region 3140p1 and the second connection region 3140p2 on the side portions 3143 formed on opposite sides of the body 3142, and connecting the first forward-moving wire 3111 and the second forward-moving wire 3112 to the first connection region 3140p1 and the second connection region 3140p2, the first forward-moving wire 3111 and the second forward-moving wire 3112 can be pulled from opposite sides of the body 3142, and also from symmetrical positions, so that the forward movement of the operation member 3140 can be precisely controlled.

In an optional embodiment, a connection region for the backward-moving wire may be formed in a rear side region of the body 3142.

FIG. 27 is a schematic perspective view illustrating a portion of the end tool of FIG. 16. FIG. 28 is a front view of FIG. 27 viewed in one direction.

Referring to FIGS. 27 and 28, the first fixed pulley 3121 and the second fixed pulley 3122 may be disposed to have an inclined shape without being positioned in parallel to each other. For example, as a specific example, based on the drawings, the first fixed pulley 3121 and the second fixed pulley 3122 may be positioned such that a gap therebetween decreases in a direction away from the second jaw 3102 (refer to FIG. 16).

Specifically, the first fixed pulley 3121 and the second fixed pulley 3122 may be positioned to face each other in the front space 3101c positioned further forward than the cartridge accommodation part 3101a of the first jaw 3101, and may be positioned symmetrically to each other as a specific example. In addition, the first fixed pulley 3121 and the second fixed pulley 3122 may be formed to have the same size.

Two outer side surfaces of the front space 3101c include the first side surface 3101t1 and the second side surface 3101t2, and the first fixed pulley 3121 and the second fixed pulley 3122 may be disposed to correspond to the first side surface 3101t1 and the second side surface 3101t2, respectively.

Each of the first side surface 3101t1 and the second side surface 3101t2 may be formed to have an inclined shape. For example, the first side surface 3101t1 and the second side surface 3101t2 may be shaped such that a gap therebetween decreases toward downward, rather than being parallel to each other with a uniform gap. As a specific example, the gap between the first side surface 3101t1 and the second side surface 3101t2 may be configured to be gradually decrease in a direction away from the second jaw 3102 (refer to FIG. 16). In addition, the first side surface 3101t1 and the second side surface 3101t2 may have shapes symmetrical to each other.

The first forward-moving wire 3111 and the second forward-moving wire 3112 may be correspondingly wound around the first fixed pulley 3121 and the second fixed pulley 3122, and the regions of the first forward-moving wire 3111 and the second forward-moving wire 3112 emerging from being wound around the lower sides of the first fixed pulley 3121 and the second fixed pulley 3122 may be directed to the first connection region 3140p1 and the second connection region 3140p2 of the operation member 3140 described above, respectively.

With such a shape, the balanced arrangement of the first and second fixed pulleys 3121 and 3122 and the first and second forward-moving wires 3111 and 3112 relative to the moving direction of the operation member 3140 may be enhanced, allowing for easier implementation of a symmetrical shape. In addition, shaking or rotational moment generated when pulling the first forward-moving wire 3111 and the second forward-moving wire 3112 may be reduced, thereby minimizing or preventing shaking of the end tool 3100.

In addition, the end tool 3100 may be shaped to reduce a width of, for example, one side of the first jaw 3101, which forms one side of the jaw 3103, specifically a width of a main region on the lower side, thereby allowing the end tool 3100 to be implemented with a compact overall structure.

Further, the first fixed pulley 3121 and the second fixed pulley 3122 may be disposed to have an inclined shape without being positioned in parallel to each other, which allows the two forward-moving wires 3111 and 3112 to emerge from being wound around the lower sides of the first fixed pulley 3121 and the second fixed pulley 3122 and be connected to the operation member 3140, i.e., positioned in a lower side region of the cartridge 3500 after passing through the first path 3101w1 and the second path 3101w2. Another regions of the two forward-moving wires 3111 and 3112 may emerge from being wound around the upper sides of the first fixed pulley 3121 and the second fixed pulley 3122 and may be respectively positioned on opposite sides of the cartridge 3500, i.e., in spaces between the cartridge 3500 and both side surfaces of the first jaw 3101.

By arranging the two forward-moving wires 3111 and 3112 in this manner, the forward-moving wires 3111 and 3112 are avoided from being fixed to the center line of the operation member 3140, thereby allowing the operation member 3140 to be implemented in a compact form while maintaining durability.

In addition, by arranging the two forward-moving wires 3111 and 3112 correspondingly to the first fixed pulley 3121 and the second fixed pulley 3122 formed in an inclined shape, such as a symmetrical shape, maximum tensions applied to the two forward-moving wires 3111 and 3112 can be equalized or made almost similar to each other, this may enhance the fatigue life of each wire.

In addition, one regions of the two forward-moving wires 3111 and 3112 can be controlled to symmetrically pass through the lower side of the cartridge 3500, and another regions thereof can be controlled to symmetrically pass through opposite sides of the cartridge 3500, and accordingly, unwanted moment or rotational forces generated when pulling the two forward-moving wires 3111 and 3112 may be reduced or prevented from causing unintended movement or shaking of the end tool 3100 or the surgical instrument 3000 including the same.

Meanwhile, referring to FIG. 28, the first fixed pulley 3121 and the second fixed pulley 3122 may be disposed in a slanted shape, i.e., an inclined shape, without being disposed in parallel to each other.

For example, each of the first fixed pulley 3121 and the second fixed pulley 3122 may be disposed to be inclined at an angle of 35 degrees (deg) to 51 degrees (deg) relative to a vertical line (a line parallel to a z-axis) based on FIG. 28.

Further, in other words, a central axis of the first fixed pulley 3121 and a central axis of the second fixed pulley 3122 may be inclined at an angle without being parallel to each other, and for example, the central axis of the first fixed pulley 3121 and the central axis of the second fixed pulley 3122 may be disposed to be inclined at an angle of 78 degrees to 110 degrees.

Further, in other words, as shown in FIG. 28, the first fixed pulley 3121 and the second fixed pulley 3122 may be shaped to be inclined, forming an adjacent angle DG.

The adjacent angle DG may range from 70 degrees (deg) to 102 degrees (deg).

When the adjacent angle DG is less than 70 degrees or more than 102 degrees, a diameter of each of the first fixed pulley 3121 and the second fixed pulley 3122 accommodated in a predetermined space of the first jaw 3101 becomes smaller, which causes the stress applied on the forward-moving wires 3111 and 3112 to be increased, and the maximum allowable tension and durability of the forward-moving wires 3111 and 3112 to be reduced, and thus, the adjacent angle DG may range from 70 degrees (deg) to 102 degrees (deg), in a specific example, the adjacent angle DG may be 86 degrees (deg). Meanwhile, the adjacent angle DG is applicable to embodiments to be described below without change.

The arrangement relationship of the operation member 3140, the plurality of forward-moving wires 3110, and the plurality of fixed pulleys 3120 will now be further described.

FIG. 29 is a schematic plan view for describing the operation member, the fixed pulley, and the forward-moving wire of the end tool of FIG. 16. FIG. 30 is a schematic perspective view for describing the operation member, the fixed pulley, and the forward-moving wire of the end tool of FIG. 16.

As described above, the first fixed pulley 3121 and the second fixed pulley 3122 are positioned in the front space 3101c of the first jaw 3101, and each of the first fixed pulley 3121 and the second fixed pulley 3122 may be fixed to the first jaw 3101 and immobile, or fixed so as to be rotatable about one shaft.

The first forward-moving wire 3111 and the second forward-moving wire 3112 may be wound around outer circumferential surfaces of the first fixed pulley 3121 and the second fixed pulley 3122, respectively, and a groove may be formed in each of the outer circumferential surfaces of the first fixed pulley 3121 and the second fixed pulley 3122.

The first fixed pulley 3121 and the second fixed pulley 3122 may be disposed closer to the distal end 3101d of the first jaw 3101 at least than the operation member 3140.

The first forward-moving wire 3111 may extend to have a length in the longitudinal direction of the first jaw 3101, and one end portion region thereof may extend to the proximal end 3101p of the first jaw 3101, and pass through the switching pulley shafts AX1 and AX2 or switching pulleys coupled thereto and the pulley shafts JX1, JX2, JX3, JX4, and JX5 or pulleys coupled thereto to be connected to the inside of the driving part, such as the manipulation part 3200 (refer to FIG. 15), so that the first forward-moving wire 3111 can be pulled through manipulation of the manipulation part 3200.

Another end portion of the first forward-moving wire 3111 may extend in a direction toward the distal end 3101d of the first jaw 3101 along the longitudinal direction of the first jaw 3101, emerge from the lower side of the first fixed pulley 3121 after being wound around the upper side of the first fixed pulley 3121 while coming into contact therewith, and extend in a direction toward proximal end 3101p of the first jaw 3101 to be connected and fixed to the first connection region 3140p1 of the operation member 3140.

In an optional embodiment, among the regions of the first forward-moving wire 3111, the region extending to the distal end 3101d of the first jaw 3101 and being directed to the upper side of the first fixed pulley 3121 may be parallel to the region emerging from the lower side of the first fixed pulley 3121, extending toward the proximal end 3101p of the first jaw 3101, and being directed toward the first connection region 3140p1 of the operation member 3140.

Accordingly, a pulling force can be effectively transmitted to the operation member 3140 when pulling the first forward-moving wire 3111 toward the proximal end 3101p. In addition, as an example, when pulling the first forward-moving wire 3111 toward the proximal end 3101p, a ratio of the pulling force to the corresponding forward-moving distance of the operation member 3140 may be precisely controlled at a predetermined ratio. As a specific example, when pulling the first forward-moving wire 3111 toward the proximal end 3101p, the ratio of the pulling force to the forward-moving distance of the operation member 3140 may be easily controlled to a value equal to, or substantially equal or similar to 1:1.

The second forward-moving wire 3112 may extend to have a length in the longitudinal direction of the first jaw 3101, and one end portion region thereof may extend to the proximal end 3101p of the first jaw 3101, and pass through the switching pulley shafts AX1 and AX2 or switching pulleys coupled thereto and the pulley shafts JX1, JX2, JX3, JX4, and JX5 or pulleys coupled thereto to be connected to the inside of the driving part, such as the manipulation part 3200 (refer to FIG. 15), so that the second forward-moving wire 3112 can be pulled through manipulation of the manipulation part 3200.

Another end portion of the second forward-moving wire 3112 may extend in a direction toward the distal end 3101d of the first jaw 3101 along the longitudinal direction of the first jaw 3101, emerge from the lower side of the second fixed pulley 3122 after being wound around the upper side of the second fixed pulley 3122 while coming into contact therewith, and extend in a direction toward proximal end 3101p of the first jaw 3101 to be connected and fixed to the second connection region 3140p2 of the operation member 3140.

In an optional embodiment, among the regions of the second forward-moving wire 3112, the region extending to the distal end 3101d of the first jaw 3101 and being directed to the upper side of the second fixed pulley 3122 may be parallel to the region emerging from the lower side of the second fixed pulley 3122, extending toward the proximal end 3101p of the first jaw 3101, and being directed toward the second connection region 3140p2 of the operation member 3140.

Accordingly, a pulling force can be effectively transmitted to the operation member 3140 when pulling the second forward-moving wire 3112 toward the proximal end 3101p.

As shown in FIGS. 29 and 30, when the first forward-moving wire 3111 and the second forward-moving wire 3112 are pulled in a first direction D1, one region of each of the first forward-moving wire 3111 and the second forward-moving wire 3112 may move in the first direction D1, and accordingly, the region of the forward-moving wire 3110 emerging from the lower sides of the first fixed pulley 3121 and the second fixed pulley 3122 after being wound around the upper sides thereof may move in a second direction D2, which is the opposite direction of the first direction D1. Accordingly, forces of the first forward-moving wire 3111 and the second forward-moving wire 3112 may be transmitted to the first connection region 3140p1 and the second connection region 3140p2 connected to the first forward-moving wire 3111 and the second forward-moving wire 3112, and the forces cause the operation member 3140 also move in a direction K1, which is the same direction as the second direction D2, i.e., move forward.

FIG. 31 and FIGS. 32A to 32C are schematic views for describing an operation of the operation member of the end tool of FIG. 16.

Referring to FIG. 31 and FIGS. 32A to 32C, for convenience of description, the first jaw 3101 is excluded, and the first forward-moving wire 3111, the second forward-moving wire 3112, the first fixed pulley 3121, the second fixed pulley 3122, and the operation member 3140 are illustrated.

Based on FIG. 31, the operation member 3140 may move in a leftward direction, i.e., move forward toward the distal end 3101d, and this forward movement is illustrated sequentially in FIGS. 32A, 32B, and 32C.

As shown in FIG. 32A, when the first forward-moving wire 3111 and the second forward-moving wire 3112 are pulled in the first direction D1, one region of each of the first forward-moving wire 3111 and the second forward-moving wire 3112 is pulled in the first direction D1, and thus the regions of the first forward-moving wire 3111 and the second forward-moving wire 3112 emerging from the lower sides of the first fixed pulley 3121 and the second fixed pulley 3122 after being wound around the upper sides thereof move in the second direction D2, which is the opposite direction of the first direction D1. Accordingly, forces of the first forward-moving wire 3111 and the second forward-moving wire 3112 are transmitted respectively to the first connection region 3140p1 and the second connection region 3140p2 respectively connected to the first forward-moving wire 3111 and the second forward-moving wire 3112, and the forces cause the operation member 3140 to move in the direction K1, which is the same direction as the second direction D2, i.e., move forward, thereby positioning the operation member 3140 in an advanced position shown in FIG. 32B.

Thereafter, as shown in FIG. 32B, when the first forward-moving wire 3111 and the second forward-moving wire 3112 are pulled further in the first direction D1, one region of the first forward-moving wire 3111 and the second forward-moving wire 3112 is pulled further in the first direction D1, and thus the regions of the first forward-moving wire 3111 and the second forward-moving wire 3112 emerging from the lower sides of the first fixed pulley 3121 and the second fixed pulley 3122 after being wound around the upper sides thereof move further in the second direction D2, which is the opposite direction of the first direction D1. Accordingly, forces of the first forward-moving wire 3111 and the second forward-moving wire 3112 are transmitted respectively to the first connection region 3140p1 and the second connection region 3140p2 connected to the first forward-moving wire 3111 and the second forward-moving wire 3112, and the forces cause the operation member 3140 to move further in the direction K1, which is the same direction as the second direction D2, i.e., move forward to a further advanced position than in FIG. 32B, thereby positioning the operation member 3140 in the further advanced position shown in FIG. 32C.

The cartridge 3500 accommodated in the end tool 3100 of FIG. 16 and a stapling motion will now be described in more detail.

FIG. 33 is a perspective view illustrating the first jaw and the cartridge of the surgical instrument of FIG. 15.

Referring to FIGS. 15 to 18, 33, and the like, the cartridge 3500 may be positioned in the first jaw 3101, and for example, the cartridge 3500 may be positioned by being coupled to the cartridge accommodation part 3101a of the first jaw 3101. For example, the cartridge 3500 may be integrally formed with the first jaw 3101 while the operation member 3140 is positioned in the first jaw 3101. In addition, in an optional embodiment, the cartridge 3500 may be configured to be mountable to and dismountable from the first jaw 3101.

The cartridge 3500 includes a plurality of staples 3530 therein to perform suturing of tissue, and performs cutting through the operation member 3140. Here, the cartridge 3500 may include a cover 3510, the staples 3530, and the withdrawal members 3535.

The cover 3510 may be configured to cover an upper portion of the cartridge accommodation part 3101a of the first jaw 3101. Staple holes 3510s through which the plurality of staples 3530 may be ejected to the outside may be formed in the cover 3510. As the staples 3530, which are accommodated inside the cartridge accommodation part 3101a before a stapling operation, are pushed and raised upward by the operation member 3140 during a stapling motion, and pass through the staple holes 3510s of the cover 3510 to be withdrawn to the outside of the cartridge 3500, stapling may be performed.

Meanwhile, a slit 3510w may be formed in the cover 3510 along a longitudinal direction of the cover 3510. The blade region 3142a of the body 3142 of the operation member 3140 may protrude out of the cartridge 3500 through the slit 3510w. As the blade of the body 3142 of the operation member 3140 passes along the slit 3510w, staple-completed tissue may be cut.

In an optional embodiment, the cartridge 3500 may include a case 3520, and the cartridge 3500 may be positioned in the case 3520 after the case 3520 is positioned in the cartridge accommodation part 3101a of the first jaw 3101.

The plurality of staples 3530 may be positioned inside the cartridge accommodation part 3101a of the first jaw 3101. As the operation member 3140 linearly moves in one direction, the plurality of staples 3530 are sequentially pushed and raised from the inside of the cartridge accommodation part 3101a of the first jaw 3101 to the outside, thereby performing suturing, that is, stapling. Here, the staples 3530 may include a material that is durable and does not have an abnormal effect on the human body, such as titanium, stainless steel, or the like.

Meanwhile, the withdrawal members 3535 may be further disposed between the cartridge accommodation part 3101a of the first jaw 3101 and the staples 3530. In other words, it may be said that the staple 3530 is positioned above the withdrawal member 3535. In this case, the operation member 3140 linearly moves in one direction to push and raise the withdrawal member 3535, and the withdrawal member 3535 may push and raise the staple 3530.

As such, the operation member 3140 may be described as pushing and raising the staples 3530 in both the case in which the operation member 3140 directly pushes and raises the staples 3530 and the case in which the operation member 3140 pushes and raises the withdrawal members 3535 and the withdrawal members 3535 pushes and raises the staples 3530 (i.e., the operation member 3140 indirectly pushes and raises the staples 3530).

As described above, the operation member 3140 may be positioned inside the cartridge accommodation part 3101a of the first jaw 3101. In addition, the operation member 3140 may include the wedge WDG or may be used in conjunction with wedge WDG, and when the operation member 3140 moves, the wedge WDG may move together therewith so that the wedge WDG may directly push and raise the staple 3530, or the wedge WDG may push and raise the withdrawal member 3535 to push and raise the staple 3530.

As described above, the movement of the first forward-moving wire 3111 and the second forward-moving wire 3112, i.e., the pulling of the first forward-moving wire 3111 and the second forward-moving wire 3112 allows the operation member 3140 connected thereto to move forward toward the distal end 3101d of the first jaw 3101.

The forward movement of the operation member 3140 may cause the wedge WDG to push and raise the withdrawal member 3535, which may also cause the staple 3530 to rise, and at the same time, cutting using the blade region 3142a of the operation member 3140 may be performed. In addition, in an optional embodiment, in the case of the end tool in which the backward-moving wire BRW is connected to the operation member 3140, the backward-moving wire BRW may be pulled to cause the operation member 3140 to move toward the proximal end 3101p of the first jaw 3101.

FIGS. 34 and 35 are views for describing a switching pulley, a yaw pulley, and a pitch pulley of the end tool of the surgical instrument of FIG. 15.

As described above, the end tool 3100 may be connected to the connection part 3400, and the end tool 3100 may rotationally move around one shaft and also rotationally move around another shaft with respect to the connection part 3400.

For example, the end tool 3100 may perform a pitch motion, i.e., a vertical rotational motion based on FIGS. 16 and 33, and the end tool 3100 may perform a yaw motion, i.e., a horizontal rotational motion based on FIGS. 16 and 33. A rotation shaft of the pitch motion and a rotation shaft of the yaw motion may be positioned in directions that may intersect or be perpendicular to each other.

As an example, the end tool 3100 may include one or more members, such as joint members, that connect the jaw 3103 to the connection part 3400, and may include the end tool hub 3108 and the pitch hub 3107.

The end tool hub 3108 may be disposed to connect the end tool 3100 to the straight part 3401 of the connection part 3400. As an example, the end tool hub 3108 may have the pulley shaft JX4 corresponding thereto, and the pulley shaft JX4 may be a rotation shaft of the pitch motion. As a specific example, the end tool 3100 may rotationally move around the pulley shaft JX4, the pitch hub 3107 may rotationally move around the pulley shaft JX4, and the jaw 3103 may be connected to the pitch hub 3107 to perform a rotational motion, i.e., a pitch motion, around the pulley shaft JX4 integrally with the pitch hub 3107.

The pitch hub 3107 is connected to the end tool hub 3108 and the jaw 3103, and may rotationally move around the pulley shaft JX4 by being axially coupled to the end tool hub 3108 by the pulley shaft JX4. Further, the jaw 3103 may be axially coupled to the pitch hub 3107 with respect to one pulley shaft, i.e., the pulley shaft JX1. The jaw 3103 may perform a rotational motion, i.e., a yaw motion, around one pulley shaft, i.e., the pulley shaft JX1 while connected to the pitch hub 3107.

An auxiliary pulley shaft may be additionally positioned together with these rotation shafts, i.e., the pulley shafts for joint motion of the end tool 3100, such as the pulley shaft JX4 for pitch motion and the pulley shaft JX1 for yaw motion.

For example, the pulley shaft JX2 different from the pulley shaft JX1 is positioned in the pitch hub 3107 to be adjacent to and parallel to the pulley shaft JX1. The pulley shaft JX2 may have an axis oriented parallel to the pulley shaft JX1, and may be positioned further away from the operation member 3140 than the pulley shaft JX1, i.e., closer to the connection part 3400.

In addition, the pulley shaft JX3 may be disposed adjacent to the pulley shaft JX4. In addition, the pulley shaft JX5 may be further disposed.

For example, the pulley shaft JX3 and the pulley shaft JX5 may be positioned on opposite sides of the pulley shaft JX4 interposed therebetween, and the pulley shaft JX3 and the pulley shaft JX5 may have axes oriented parallel to the pulley shaft JX4.

As a specific example, the pulley shaft JX3 may be positioned between the pulley shaft JX2 and the pulley shaft JX4, and may have an axis intersecting or orthogonal to the pulley shaft JX2 and the pulley shaft JX1. The pulley shaft JX5 may be positioned further away from the operation member 3140 than the pulley shaft JX4, i.e., closer to the connection part 3400.

One or more switching pulley shafts AX1 and AX2, i.e., a first switching pulley shaft AX1 and a second switching pulley shaft AX2, may be disposed closer to the operation member 3140 than the pulley shafts JX1, JX2, JX3, JX4, and JX5

The first switching pulley shaft AX1 and the second switching pulley shaft AX2 may be shafts formed parallel to each other. The first switching pulley shaft AX1 and the second switching pulley shaft AX2 may be sequentially disposed in a direction toward the distal end 3101d of the first jaw 3101 so as to be offset from each other based on a width direction of the first jaw 3101, and may be disposed with some regions overlapping.

One or more pulleys may be disposed on the pulley shafts JX1, JX2, JX3, JX4, and JX5 and the switching pulley shafts AX1 and AX2.

When description is given in the order from the proximal end of the first jaw 3101 towards the connection part 3400, one or more switching pulleys AXP1 corresponding to the first switching pulley shaft AX1 and one or more switching pulleys AXP2 corresponding to the second switching pulley shaft AX2 are positioned.

The one or more switching pulleys AXP1 corresponding to the first switching pulley shaft AX1, and the one or more switching pulleys AXP2 corresponding to the second switching pulley shaft AX2 may all be positioned in a place overlapping the first jaw 3101, for example, may be positioned in one region of the coupling region 3101z of the first jaw 3101 so as not to overlap the pitch hub 3107.

The first forward-moving wire 3111 and the second forward-moving wire 3112 may come into contact with the switching pulleys AXP1 and the switching pulley AXP2, respectively, in at least one region, and thus may be guided in path.

For example, the first forward-moving wire 3111 may enter the switching pulleys AXP1 from the outside, wind inward, continue winding into one region of the switching pulleys AXP2 adjacent to the switching pulleys AXP1, and then exit from the switching pulleys AXP2.

The second forward-moving wire 3112 may come into contact with the switching pulleys AXP2 in at least one region, and may be guided in path. The first forward-moving wire 3111 and the second forward-moving wire 3112, which are collected in the switching pulley AXP2, may be wound together around one or more pulleys JXP1 corresponding to the pulley shaft JX1, which will be described below, in the same direction.

Accordingly, the first forward-moving wire 3111 and the second forward-moving wire 3112 can be moved in one direction, and can be organized on one side of the shaft and pulley for each joint motion, such as the pulley shaft and pulleys for pitch motion and the pulley shaft and pulleys for yaw motion, rather than on opposite sides of the shaft and pulley for each joint motion, thus facilitating precise implementation of simultaneous and straightforward control over the first forward-moving wire 3111 and the second forward-moving wire 3112.

For example, when the end tool 3100 performs yaw and pitch motions, the wires wound around the yaw pulley (for yaw motion) or the pitch pulley (for pitch motion) may be wound or unwound further to one side, whereas the first forward-moving wire 3111 and the second forward-moving wire 3112 of the present embodiment can be gathered and organized on one side, i.e., wound in the same direction on the yaw pulley and the pitch pulley, and thus may be wound or unwound the same distance. Accordingly, the two wires can be easily controlled together by a single driving unit (e.g., an actuator) since the first forward-moving wire 3111 and the second forward-moving wire 3112 need only be wound or unwound by the same distance in order to make yaw and/or pitch manipulation to prevent the operation member 3140 from moving.

In an optional embodiment, the first forward-moving wire 3111 and the second forward-moving wire 3112 may be wound in different directions around the yaw pulley and the pitch pulley, respectively. When the first forward-moving wire 3111 or the second forward-moving wire 3112 is pulled, an undesired rotational force may be generated in the end tool 3100, which may occur because each of the first forward-moving wire 3111 and the second forward-moving wire 3112 is offset by a radius of the yaw pulley or the pitch pulley, causing asymmetrical forces to be applied to the end tool 3100. At this point, when the first forward-moving wire 3111 and the second forward-moving wire 3112 are wound in opposite directions around the yaw pulley and the pitch pulley, respectively, the rotational forces in the end tool 3100 may be canceled through application of symmetrical forces, thereby reducing a change in position/posture of the end tool 3100 during a stapling process. In this case, the first forward-moving wire 3111 and the second forward-moving wire 3112 may be wound around one side and unwound around another side, and thus a compensating member may be further disposed to compensate for this. As an example, the first forward-moving wire 3111 and the second forward-moving wire 3112 may be formed into a single loop by placing a common pulley in one region of the end tool 3100, or the connection part 3400 or the manipulation part 3200 adjacent thereto. As another example, the first forward-moving wire 3111 and the second forward-moving wire 3112 may each be manipulated by a separate driving unit (e.g., an actuator).

In order to facilitate the path guidance for the first forward-moving wire 3111 and the second forward-moving wire 3112, the switching pulley AXP1 and the switching pulley AXP2 may have structures symmetrical to each other with respect to an extension line of the operation member 3140, that is, may have structures offset by the same distance with respect to the extension line of the operation member 3140. This allows the size of each of the switching pulley AXP1 and the switching pulley AXP2 to be increased, thereby improving the efficiency and stability of the path guidance for the first forward-moving wire 3111 and the second forward-moving wire 3112.

One or more pulleys JXP1 are disposed to correspond to the pulley shaft JX1, and one or more pulleys JXP2 corresponding to the pulley shaft JX2 are disposed adjacent to the one or more pulleys JXP1. The pulley JXP1 and the pulley JXP2 may have axes parallel to each other.

For example, the pulley JXP1 and the pulley JXP2 are positioned in the first hub 3107a (refer to FIG. 19) of the pitch hub 3107. One or more pulleys JXP2 guide the paths along which the wires, which are disposed to correspond to one or more pulleys JXP1, are driven, ensuring that the wires have a clear path to the pulley shaft JX4, or more closely, to the pulley shaft JX3 and a pulley JXP3 corresponding thereto.

Further, one or more pulleys JXP3 are disposed to correspond to the pulley shaft JX3, and one or more pulleys JXP4 corresponding to the pulley shaft JX4 are positioned adjacent to the one or more pulleys JXP4. For example, the pulley JXP3 and the pulley JXP4 are positioned in the second hub 3107b (refer to FIG. 19) of the pitch hub 3107. In addition, one or more pulleys JXP5 may be disposed to correspond to the pulley shaft JX5. The pulley JXP3, the pulley JXP4, and the pulley JXP5 may have axes that are parallel to each other and intersect or orthogonal the axes of the pulley JXP1 and the pulley JXP2.

Meanwhile, by precisely controlling the paths of the first forward-moving wire 3111 and the second forward-moving wire 3112 as shown in FIGS. 34 and 35, the drive efficiency and control characteristics of the operation member 3140 can be maximized through the first forward-moving wire 3111 and the second forward-moving wire 3112.

As described above, the switching pulley AXP1 and the switching pulleys AXP2 are positioned further forward than the pulley shaft JX4 and the pulley shaft JX1, which are respectively for two joint motions such as a pitch motion and a yaw motion of the end tool 3100, i.e., disposed closer to the operation member 3140 than the above pulleys.

As a specific example, the switching pulley AXP1 and the switching pulleys AXP2 are positioned further forward than the pulley JXP4 corresponding to the pulley shaft JX4 for a pitch motion, the pulley JXP1 corresponding to the pulley shaft JX1 for a yaw motion, the pulley JXP3 and the pulley JXP5 corresponding to pitch auxiliary pulleys, and the pulley JXP2 that is a yaw auxiliary pulley, i.e., disposed closer to the operation member 3140 than the above pulleys.

As a result, the switching pulley AXP1 and the switching pulleys AXP2 are positioned further forward than the pulley JXP1, the pulley JXP2, the pulley JXP3, the pulley JXP4, and the pulley JXP5, i.e., disposed closer to the operation member 3140 than the above pulleys.

Accordingly, the first forward-moving wire 3111 may enter the switching pulleys AXP1 from the outside, wind inward, continue winding into one region of the switching pulleys AXP2 adjacent to the switching pulleys AXP1, and then exit from the switching pulleys AXP2. In addition, the first forward-moving wire 3111 and the second forward-moving wire 3112 may be gathered together on one side (e.g., an outer side) of the switching pulley AXP2.

In addition, the first forward-moving wire 3111 and the second forward-moving wire 3112, which are gathered together on the outer side of the switching pulley AXP2, may be directed to the connection part 3400 after being simultaneously rerouted at an outer side of the pulley JXP1 corresponding to the pulley shaft JX1, which is a yaw pulley shaft, being wound around the pulley JXP2 corresponding to the pulley shaft JX2, which is a yaw auxiliary pulley shaft, to be rerouted, being controlled in path height by the pulley JXP3 corresponding to the pulley shaft JX3, being stably positioned on a lower side of the pulley JXP4 corresponding to the pulley shaft JX4, which is a pitch shaft, and passing through the pulley JXP5.

That is, by first gathering the first forward-moving wire 3111 and the second forward-moving wire 3112 together through the switching pulley AXP1 and the switching pulley AXP2, the paths of the first forward-moving wire 3111 and the second forward-moving wire 3112 can be easily guided by simultaneously corresponding to the rotation shafts and the pulleys and their auxiliary pulleys for the joint motion of the end tool 3100, so that the accuracy and stability of the forward movement of the operation member 3140 can be improved.

In some embodiments, by controlling heights of the paths of the first forward-moving wire 3111 and the second forward-moving wire 3112, which are gathered together, using the pulley JXP3 prior to directing to the pulley JXP4 corresponding to the pulley shaft JX4, which is a pitch shaft, the first forward-moving wire 3111 and the second forward-moving wire 3112 can be stably wound around the pulley JXP4 corresponding to the pulley shaft JX4, which is a pitch shaft, and the freedom of size, design and arrangement of the pulley JXP4 can be improved.

While each of the embodiments described above may be implemented independently, it goes without saying that the structures of the respective embodiments may also be applied in combination.

According to the present disclosure, stability during stapling procedures performed using a surgical instrument can be improved.

The present disclosure has been described above in relation to its preferred embodiments. It will be understood by those skilled in the art that various changes in form and details may be made therein without departing from the essential features of the present disclosure. Therefore, the disclosed embodiments should be considered in descriptive sense only and not for purposes of limitation. The scope of the present disclosure is defined not by the detailed description of the disclosure but by the appended claims, and all differences within the scope will be construed as being included in the present disclosure.

What is claimed is:

1. An end tool comprising:
a first jaw configured to be rotatable around a pitch rotation axis and including a cartridge accommodation part configured to accommodate a cartridge therein;
a second jaw facing the first jaw and configured to rotate with respect to the first jaw; and
the cartridge accommodated in the cartridge accommodation part and including a plurality of staples,
wherein the second jaw includes a guard part extending toward the first jaw, such that at least one region of the guard part is positioned in the cartridge accommodation part during the rotation of the second jaw with respect to the first jaw.

2. The end tool of claim 1, wherein
the first jaw further includes side wall parts extending from opposite sides of a base of the first jaw toward the second jaw, and
the side wall parts form the cartridge accommodation part in a space therebetween.

3. The end tool of claim 2, wherein the guard part is positioned between the cartridge and the side wall parts when the cartridge is accommodated in the cartridge accommodation part.

4. The end tool of claim 2, wherein
the guard part comprises a first guard part and a second guard part,
the first guard part and the second guard part are disposed to face each other on opposite sides of the second jaw with respect to a longitudinal axis of the second jaw, and
a width between the first guard part and the second guard part is less than a width between the side wall parts.

5. The end tool of claim 2, wherein a width between the side wall parts is constant in a direction in which the first jaw extends.

6. The end tool of claim 2, wherein the guard part is positioned closer to the cartridge than to the side wall parts when the cartridge is accommodated in the cartridge accommodation part.

7. The end tool of claim 1, wherein the guard part is positioned on a proximal side of the second jaw.

8. The end tool of claim 1, wherein at least one region of the guard part is positioned in the cartridge accommodation part when the first jaw and the second jaw are spaced apart at the greatest distance.

9. The end tool of claim 1, further comprising a wire positioned in the cartridge accommodation part in a longitudinal direction of the first jaw,
wherein the guard part is positioned between the cartridge and the wire when the cartridge is accommodated in the cartridge accommodation part.

10. The end tool of claim 1, wherein
the second jaw further includes a hinge part having a first rotation shaft serving as a center of the rotation with respect to the first jaw, and
the guard part is configured to cover at least a portion of outer side of the hinge part.

11. The end tool of claim 1, wherein the guard part restricts a body tissue from being inserted into a predefined region when the body tissue is clamped between the first jaw and the second jaw.

12. The end tool of claim 1, wherein
in a closed state of the second jaw, in which the second jaw is rotated to be in closest proximity to the first jaw,
a width of the first jaw defines a maximum outer diameter of the end tool, and
a width of the second jaw is less than the maximum outer diameter of the end tool.

13. The end tool of claim 12, wherein
the guard part comprises a first guard part and a second guard part,
the width of the first jaw is greater than a distance between the first guard part and the second guard part in at least one region.

14. The end tool of claim 12, wherein, in the closed state of the second jaw, the guard part is restricted from coming into contact with an outside by the first jaw.

15. The end tool of claim 1, wherein
in a closed state in which the first jaw and the second jaw are rotated to be in closest proximity to each other,
a maximum outer diameter of the end tool is defined as a maximum distance between the first jaw and the second jaw in a cross section perpendicular to the longitudinal axis of the end tool, and
the guard part is positioned in a space defined by the maximum outer diameter of the end tool.

16. A surgical instrument comprising:
a connection part;
an end tool disposed on a first side of the connection part and rotatable in at least one direction; and
a manipulation part disposed on a second side of the connection part and configured to control a rotation of the end tool;
wherein the end tool includes:
a first jaw configured to be rotatable around a pitch rotation axis and including a cartridge accommodation part configured to accommodate a cartridge therein;
a second jaw facing the first jaw and configured to rotate with respect to the first jaw; and
the cartridge accommodated in the cartridge accommodation part and including a plurality of staples,
wherein the second jaw includes a guard part extending toward the first jaw, such that at least one region of the guard part is positioned in the cartridge accommodation part during the rotation of the second jaw with respect to the first jaw.

17. The surgical instrument of claim 16, wherein
the first jaw further includes side wall parts extending from opposite sides of a base of the first jaw toward the second jaw, and
the side wall parts form the cartridge accommodation part in a space therebetween.

18. The surgical instrument of claim 17, wherein the guard part is positioned between the cartridge and the side wall parts when the cartridge is accommodated in the cartridge accommodation part.

19. The surgical instrument of claim 16, wherein
in a closed state of the second jaw, in which the second jaw is rotated to be in closest proximity to the first jaw,
a width of the first jaw defines a maximum outer diameter of the end tool, and
a width of the second jaw is less than the maximum outer diameter of the end tool.

20. The surgical instrument of claim 17, wherein
in a closed state in which the first jaw and the second jaw are rotated to be in closest proximity to each other,
a maximum outer diameter of the end tool is defined as a maximum distance between the first jaw and the second jaw in a cross section perpendicular to the longitudinal axis of the end tool, and
the guard part is positioned in a space defined by the maximum outer diameter of the end tool.
